(19) United States
(12) Patent Application Publication
(10) Pub. No.: US 2024/0292858 A1
(43) Pub. Date: Sep. 5, 2024

(54) METHOD FOR PRODUCING A COFFEE EXTRACT

(71) Applicant: Novozymes A/S, Bagsvaerd (DK)

(72) Inventors: Jens Magnus EKLOF, Hostrupsvej (DK); Louise Rasmussen, Helsinge (DK); Gitte Budolfsen Lynglev`, Frederiksberg (DK); Nikolaj Spodsberg, Holte (DK); Kristian Bertel Roemer Krogh, Bagsvaerd (DK)

(73) Assignee: Novozymes A/S, Bagsvaerd (DK)

(21) Appl. No.: 18/663,928

(22) Filed: May 14, 2024

Related U.S. Application Data
(62) Division of application No. 15/737,583, filed on Dec. 18, 2017.

(30) Foreign Application Priority Data
Jun. 26, 2015 (EP) 15174110.5
Jun. 26, 2015 (EP) 15174117.0

(51) Int. Cl. A23F 5/24; A23F 5/12; C12N 9/24

(52) U.S. Cl. CPC A23F 5/246; A23F 5/12; C12N 9/2488; C12N 9/2494

(57) ABSTRACT

The present invention relates to a method for producing a coffee extract which comprises use of an enzyme having mannanase activity. The invention also relates to polypeptides having endo-beta-1,4-mannanase activity and polynucleotides encoding the polypeptides. The invention also relates to nucleic acid constructs, vectors, and host cells comprising the polynucleotides as well as methods of producing and using the polypeptides.

### CROSS-REFERENCE TO RELATED APPLICATIONS

This application is a divisional of U.S. application Ser. No. 15/737,583 filed on Dec. 18, 2017 now pending, which is a 35 U.S.C. 371 national application of international application no. PCT/EP2016/064727 filed Jun. 24, 2016, which claims priority or the benefit under 35 U.S.C. 119 of European application nos. 15174110.5 and 15174117.0, each of which was filed on Jun. 26, 2015. The content of each application is fully incorporated herein by reference.

### REFERENCE TO A SEQUENCE LISTING

This application contains a Sequence Listing in computer readable form,-The contents of the electronic sequence listing created on May 13, 2024, named SQ.xml and 31,197 bytes in size, is hereby incorporated by reference in its entirety.

### BACKGROUND OF THE INVENTION

### Field of the Invention

The present invention relates to enzyme-assisted production of coffee extracts. The invention also relates to polypeptides having endo-beta-1,4-mannanase activity and polynucleotides encoding the polypeptides. The invention also relates to nucleic acid constructs, vectors, and host cells comprising the polynucleotides as well as methods of producing and using the polypeptides.

### Description of the Related Art

Coffee extract, i.e., an aqueous solution of soluble solids extracted from the coffee bean, has various industrial applications. It is used, e.g., in the manufacture of instant coffee; in ready-to-drink coffee products such as canned coffee and bottled coffee drinks; and in non-beverage applications such as instant desserts, confectionary products and flavours.

Commercial coffee extracts are typically produced by stagewise thermal processing, a combination of wetting, extraction and hydrolysis stages, which solubilizes a high percentage of the roast and ground coffee solids. Very high temperatures are required to effect thermal hydrolysis and this may lead to off-flavours and to cost and capital intensive processes.

Use of various different enzymes in the production of coffee extracts to improve product quality and process economics has been suggested (see, e.g., U.S. Pat. No. 4,983,408, WO2007/011531, U.S. Pat. No. 5,714,183). Use of mannanase in the production of a soluble coffee extract has been disclosed in, e.g., WO2007/011531 and U.S. Pat. No. 5,714,183.

It is an object of the present invention to obtain coffee extracts having a high yield of soluble solids.

### SUMMARY OF THE INVENTION

The present inventors have identified novel mannanase enzymes and shown that these are useful for extraction of roast and ground coffee thus giving a high yield of dry matter in the coffee extract obtained.

The present invention therefore relates to method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
wherein the enzyme having mannanase activity has at least 60% sequence identity to any of SEQ ID NO: 3, SEQ ID NO: 8, SEQ ID NO: 13 or SEQ ID NO: 18.

The inventors have further found out that thermostable mannanase enzymes are particularly useful for extraction of roast and ground coffee. The coffee extracts obtained have a high yield of dry matter. Use of a thermostable mannanase enzyme is an advantage in the production of coffee extracts since this will allow for extraction at higher temperature. In general, extraction at high temperature will give a higher yield. Also, high temperature will reduce microbial growth. Further, in the stagewise extraction process used in commercial production of coffee extracts, an extraction at very high temperature may take place immediately before an extraction wherein a mannanase enzyme is applied, and use of a thermostable mannanase will allow for less cooling between those two extractions.

In a second aspect, the present invention therefore relates to a method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
wherein the enzyme having mannanase activity is thermostable.

Preferably, the enzyme having mannanase activity has a melting temperature (Tm) determined by Differential Scanning Calorimetry (DSC) of at least 80° C., preferably at least 85° C. or at least 90° C.

Preferably, the incubation in step d. is performed at a temperature of at least 60° C. such as at least 65° C., preferably at least 70° C. such as at least 75° C. or at least 80° C.

The inventors have further found out that mannanase enzymes comprising a CBM1 binding domain are particularly useful for extraction of roast and ground coffee.

In a third aspect, the present invention therefore relates to a method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
wherein the enzyme having mannanase activity comprises a CBM1 binding domain.

In yet another aspect, the present invention relates to polypeptides having endo-beta-1,4-mannanase activity, selected from the group consisting of:
(a) a polypeptide having at least 75% sequence identity to the polypeptide of SEQ ID NO: 3;
(b) a polypeptide having at least 90% sequence identity to the polypeptide of SEQ ID NO: 8; and
(c) a polypeptide having at least 80% sequence identity to the polypeptide of SEQ ID NO: 13.

In one embodiment, the invention relates to polypeptides having endo-beta-1,4-mannanase activity, selected from the group consisting of:
(a) a polypeptide having at least 75% sequence identity to the polypeptide of SEQ ID NO: 3;
(b) a polypeptide encoded by a polynucleotide that hybridizes under low stringency conditions with (i) the mature polypeptide coding sequence of SEQ ID NO: 1, (ii) the cDNA sequence thereof, or (iii) the full-length complement of (i) or (ii);
(c) a polypeptide encoded by a polynucleotide having at least 75% sequence identity to the mature polypeptide coding sequence of SEQ ID NO: 1 or the cDNA sequence thereof;
(d) a variant of the polypeptide of SEQ ID NO: 3 comprising a substitution, deletion, and/or insertion at one or more positions; and
(e) a fragment of the polypeptide of (a), (b), (c), or (d) that has endo-beta-1,4-mannanase activity.

In another embodiment, the invention relates to polypeptides having endo-beta-1,4-mannanase activity, selected from the group consisting of:
(a) a polypeptide having at least 90% sequence identity to the polypeptide of SEQ ID NO: 8;
(b) a polypeptide encoded by a polynucleotide that hybridizes under low stringency conditions with (i) the mature polypeptide coding sequence of SEQ ID NO: 6, (ii) the cDNA sequence thereof, or (iii) the full-length complement of (i) or (ii);
(c) a polypeptide encoded by a polynucleotide having at least 90% sequence identity to the mature polypeptide coding sequence of SEQ ID NO: 6 or the cDNA sequence thereof;
(d) a variant of the polypeptide of SEQ ID NO: 8 comprising a substitution, deletion, and/or insertion at one or more positions; and
(e) a fragment of the polypeptide of (a), (b), (c), or (d) that has endo-beta-1,4-mannanase activity.

In yet another embodiment, the invention relates to polypeptides having endo-beta-1,4-mannanase activity, selected from the group consisting of:
(a) a polypeptide having at least 80% sequence identity to the polypeptide of SEQ ID NO: 13;
(b) a polypeptide encoded by a polynucleotide that hybridizes under low stringency conditions with (i) the mature polypeptide coding sequence of SEQ ID NO: 11, (ii) the cDNA sequence thereof, or (iii) the full-length complement of (i) or (ii);
(c) a polypeptide encoded by a polynucleotide having at least 80% sequence identity to the mature polypeptide coding sequence of SEQ ID NO: 11 or the cDNA sequence thereof;
(d) a variant of the polypeptide of SEQ ID NO: 13 comprising a substitution, deletion, and/or insertion at one or more positions; and
(e) a fragment of the polypeptide of (a), (b), (c), or (d) that has endo-beta-1,4-mannanase activity.

### DEFINITIONS

Mannanase: In the context of the present invention a “mannanase” is a beta-mannanase. It may be an enzyme defined according to the art as an endo-beta-1,4-mannanase (EC 3.2.1.78) which catalyses the hydrolysis of 1,4-beta-D-mannosidic linkages in mannans, galactomannans and glucomannans, which enzyme has the alternative names mannan endo-1,4-beta-mannosidase; 1,4-beta-D-mannan mannanohydrolase; endo-1,4-beta-mannanase; beta-mannanase B; beta-1,4-mannan 4-mannanohydrolase; endo-beta-mannanase; and beta-D-mannanase. For purposes of the present invention, mannanase activity may be determined using the activity assay described by Staalbrand et al. (1993), Purification and characterization of two beta-mannanases from Trichoderma reesei, J. Biotechnol., 29:229-42. In one aspect, a mannanase to be used in a method of the present invention has at least 20%, e.g., at least 40%, at least 50%, at least 60%, at least 70%, at least 80%, at least 90%, at least 95%, or at least 100% of the mannanase activity of the polypeptide of GENESEQP accession number AXU66990 shown herein as SEQ ID NO: 16.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has endo-beta-1,4-mannanase activity. In one aspect, a fragment of the polypeptide of SEQ ID NO: 3 contains at least 350 amino acid residues, at least 375 amino acid residues, or at least 400 amino acid residues. In one aspect, a fragment of the polypeptide of SEQ ID NO: 8 contains at least 300 amino acid residues, at least 315 amino acid residues, or at least 330 amino acid residues. In one aspect, a fragment of the polypeptide of SEQ ID NO: 13 contains at least 300 amino acid residues, at least 315 amino acid residues, or at least 330 amino acid residues.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide of the polypeptide of SEQ ID NO: 2 is amino acids 18-431 of SEQ ID NO: 2. In one aspect, the mature polypeptide of the polypeptide of SEQ ID NO: 7 is amino acids 18-367 of SEQ ID NO: 7. In one aspect, the mature polypeptide of the polypeptide of SEQ ID NO: 12 is amino acids 18-361 of SEQ ID NO: 12. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having endo-beta-1,4-mannanase activity. In one aspect, the mature polypeptide coding sequence of SEQ ID NO: 1 is nucleotides 52 to 1545 of SEQ ID NO: 1 or the cDNA sequence thereof. In one aspect, the mature polypeptide coding sequence of SEQ ID NO: 6 is nucleotides 52 to 1219 of SEQ ID NO: 6 or the cDNA sequence thereof. In one aspect, the mature polypeptide coding sequence of SEQ ID NO: 11 is nucleotides 52 to 1200 of SEQ ID NO: 11 or the cDNA sequence thereof.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Nucleic acid construct: The term “nucleic acid construct” means a nucleic acid molecule, either single-or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having endo-beta-1,4-mannanase activity.

Variant: The term “variant” means a polypeptide having endo-beta-1,4-mannanase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide of the polypeptide of SEQ ID NO: 2 is amino acids 18-431 of SEQ ID NO: 2. In one aspect, the mature polypeptide of the polypeptide of SEQ ID NO: 7 is amino acids 18-367 of SEQ ID NO: 7. In one aspect, the mature polypeptide of the polypeptide of SEQ ID NO: 12 is amino acids 18-361 of SEQ ID NO: 12. In one aspect, the mature polypeptide of the polypeptide of SEQ ID NO: 17 is amino acids 28-319 of SEQ ID NO: 17 (based on N-terminal sequencing and mass spectrometry (MS) of the full-length protein). It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Thermostable: In the context of the present invention, a thermostable enzyme having mannanase activity may have a melting temperature (Tₘ) determined by Differential Scanning Calorimetry (DSC) of at least 80° C., preferably at least 85° C., more preferably at least 90° C. The Tₘ may be determined as described in the Examples.

### DETAILED DESCRIPTION OF THE INVENTION

The present invention invention in one aspect relates to method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
wherein the enzyme having mannanase activity has at least 60% sequence identity to any of SEQ ID NO: 3, SEQ ID NO: 8, SEQ ID NO: 13 or SEQ ID NO: 18.

In an embodiment, the enzyme having mannanase activity has a sequence identity to the polypeptide of SEQ ID NO: 3 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In an embodiment, the enzyme differs by up to 10 amino acids, e.g., 1, 2, 3, 4, 5, 6, 7, 8, 9, or 10, from the polypeptide of SEQ ID NO: 3. In one embodiment, such enzyme is thermostable.

In one embodiment, the enzyme having mannanase activity preferably comprises or consists of the amino acid sequence of SEQ ID NO: 3 or an allelic variant thereof; or is a fragment thereof having mannanase activity. In another aspect, the enzyme comprises or consists of the amino acid sequence of SEQ ID NO: 3. In one embodiment, such enzyme is thermostable.

In an embodiment, the enzyme having mannanase activity has a sequence identity to the polypeptide of SEQ ID NO: 18 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In an embodiment, the enzyme differs by up to 10 amino acids, e.g., 1, 2, 3, 4, 5, 6, 7, 8, 9, or 10, from the polypeptide of SEQ ID NO: 18. In one embodiment, such enzyme is thermostable.

In one embodiment, the enzyme having mannanase activity preferably comprises or consists of the amino acid sequence of SEQ ID NO: 18 or an allelic variant thereof; or is a fragment thereof having mannanase activity. In another aspect, the enzyme comprises or consists of the amino acid sequence of SEQ ID NO: 18. In one embodiment, such enzyme is thermostable.

In an embodiment, the enzyme having mannanase activity has a sequence identity to the polypeptide of SEQ ID NO: 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In an embodiment, the enzyme differs by up to 10 amino acids, e.g., 1, 2, 3, 4, 5, 6, 7, 8, 9, or 10, from the polypeptide of SEQ ID NO: 8.

In one embodiment, the enzyme having mannanase activity preferably comprises or consists of the amino acid sequence of SEQ ID NO: 8 or an allelic variant thereof; or is a fragment thereof having mannanase activity. In another aspect, the enzyme comprises or consists of the amino acid sequence of SEQ ID NO: 8.

In an embodiment, the enzyme having mannanase activity has a sequence identity to the polypeptide of SEQ ID NO: 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In an embodiment, the enzyme differs by up to 10 amino acids, e.g., 1, 2, 3, 4, 5, 6, 7, 8, 9, or 10, from the polypeptide of SEQ ID NO: 13.

In one embodiment, the enzyme having mannanase activity preferably comprises or consists of the amino acid sequence of SEQ ID NO: 13 or an allelic variant thereof; or is a fragment thereof having mannanase activity. In another aspect, the enzyme comprises or consists of the amino acid sequence of SEQ ID NO: 13.

In a second aspect, the invention relates to a method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
wherein the enzyme having mannanase activity is thermostable.

The description and embodiments below is relevant for both of these aspects of the present invention.

In a preferred embodiment, the enzyme having mannanase activity has a melting temperature (Tₘ) determined by Differential Scanning Calorimetry (DSC) of at least 80° C., preferably at least 85° C., more preferably at least 90° C. The melting temperature Tₘ may be determined as described in the Examples.

In a preferred embodiment, incubation is performed at a temperature of at least 60° C. such as at least 65° C., preferably at least 70° C. such as at least 75° C. or at least 80° C.

In another preferred embodiment, incubation is performed at a temperature typically in the range of about 50° C. to about 100° C., preferably about 60° C. to about 100° C., more preferably about 70° C. to about 100° C., even more preferably about 80° C. to about 100° C.

In an embodiment, the enzyme having mannanase activity has been isolated.

In an embodiment, the enzyme having mannanase activity is an endo-beta-1,4-mannanase, preferably a GH5 endo-beta-1,4-mannanase, more preferably a GH5_7 endo-beta-1,4-mannanase or a GH5_8 endo-beta-1,4-mannanase. In a preferred embodiment, the enzyme having mannanase activity is a GH5_7 endo-beta-1,4-mannanase. In another preferred embodiment, the enzyme having mannanase activity is a GH5_8 endo-beta-1,4-mannanase.

An enzyme having mannanase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The enzyme may be a fungal enzyme. For example, the enzyme may be obtained from yeast such as from Candida, Kluyveromyces, Pichia, Saccharomyces, Schizosaccharomyces, or Yarrowia; or from a filamentous fungus such as from Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria.

In another embodiment, the enzyme is obtained from Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis.

In another embodiment, the enzyme is obtained from Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride.

In one embodiment, the enzyme is obtained from Talaromyces, e.g., from Talaromyces leycettanus.

In another embdodiment, the enzyme is obtained from Chaetomium, e.g., from Chaetomium virescens.

In another embodiment, the enzyme is obtained from Sordaria, e.g., from Sordaria macrospora.

In another embodiment, the enzyme is obtained from Caldicellulosiruptor, e.g., from Caldicellulosiruptor saccharolyticus.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

In one embodiment, the enzyme having mannanase activity is not obtained from Aspergillus niger.

The method of the present invention can be applied to fresh roast and ground coffee beans or to roasted coffee grounds which have been previously extracted with water.

In a preferred embodiment, the roast and ground coffee beans have been partially extracted.

In an embodiment, one first extraction is performed in step b.

The method of the invention can be applied to ground coffee beans obtained by conventional soluble coffee processing. Therein, roast coffee is typically ground and (thermally) extracted with water in multiple stages. A two-stage execution is typical in the art, wherein the first stage comprises wetting the coffee grounds, recovery of flavour and extraction of the readily soluble components (such as caffeine, minerals and simple sugars). The second stage is typically a hydrolysis stage, where large coffee bio-polymers and bound components are broken down to smaller water-soluble ones. In the first stage, the roast coffee is typically extracted with water at or below 100° C. The grounds from this extraction, which may be referred to as “atmospheric grounds”, are then extracted with superheated water at temperatures between 140° C. and 180° C. or even higher. The partially extracted grounds from the superheated extraction may be referred to as “super-heated grounds”.

If the method of the invention is applied to partially extracted grounds, a first extraction may be carried out by adding the roast and ground coffee which may have an average particle size of about 900 micron to a jacketed stirred tank which contains water, wherein the solids to water ratio is about 1:5. The slurry is stirred, heated indirectly to a temperature of less than about 140° C., preferably in the range of about 85-90° C., and held at this temperature for about 30 minutes. The slurry is then discharged from the vessel and the subsequent grounds and extract separated using a filter. The partially extracted grounds are subjected to a second extraction according to the invention and the extract produced in the first extraction (step b) may be blended with the second extract obtained in step e.

Also, a multi-stage execution (i.e., more than two extractions) is typical in the art. After the first stage, multiple subsequent stages are performed. The method of the invention may be part of such multi-stage extraction. Partially extracted grounds which have been subjected to one or more first extractions are subjected to an extraction according to the invention and the extract produced in the one or more previous extractions may be blended with the extract obtained in step e.

In the context of the present invention, partially extracted ground coffee beans or partially extracted coffee grounds means that the ground coffee beans have been subjected to at least one extraction. Such partially extracted ground coffee beans may also be referred to as spent coffee grounds.

The method of the invention may, in general, be applied to roast and ground coffee comprising roasted beans which were ground to an average particle size of between about 0.1 to about 5 mm, preferably between about 0.2 to about 1 mm.

In addition, a flavour management pre-treatment step can be added to the method of the invention to recover the aroma compounds or aromatic constituents of the coffee prior to the extraction and/or hydrolysis stages. Useful processes include, but are not limited to, those de-scribed in EP 0 489 401. A practical execution includes wetting roast and ground coffee with water in a vessel in a ratio of about 1:0.5 by weight. Vacuum is applied to the vessel (e.g., about 150 mbar) and then low pressure steam is applied to the bed of wetted grounds for up to about 4 to 8 minutes to evaporate aroma compounds from the roast and ground coffee. Volatile compounds drawn off are condensed, for example at about 5° C. and retained to be added back to extracts or extracted solids.

The method of the invention can be practiced on roast coffee which has been steamed-purged at low pressure to extract volatile flavour components, as described above.

The method of the invention may be applied to any type of coffee grounds with hydrolysable matter known to those skilled in the art, such as de-oiled coffee grounds, decaffeinated coffee grounds, wet-milled coffee grounds, asparaginase-treated coffee grounds, etc.

The enzymatic treatment of the roast and ground coffee beans is to be performed at a temperature where the enzymes are active and for sufficiently long time to permit enzyme reaction.

In one possible batch mode of operation, after the enzymatic reaction is essentially completed, the mixture is subjected to a gross separation, for example centrifugation or belt filtration, which removes most of the insoluble solids. The separated extract, still containing fine particulates, oil and enzyme protein, is recirculated through a cross-flow membrane device, which removes all insolubles and can also remove enzyme. Most or all of the enzyme remains in the membrane retentate and may be recycled to the reaction.

In one possible mode of operation, semi-permeable membrane permeate is constantly withdrawn during the enzyme reaction, i.e. a portion of the reaction mixture is continuously cir-culated through the cross-flow semi-permeable membrane separation cell. The process can be operated in a semi-continuous mode, wherein permeate is withdrawn until the volume in the re-action vessel diminishes to the point where its viscosity or the pressure drop becomes high. At this point, some retentate is purged and fresh coffee slurry fed and some fresh enzyme added. The purged retentate can be discarded or can be washed to recover the enzyme which is then re-used. The enzyme in the remaining (non-purged) retentate is retained and re-used.

Alternatively, fresh feed slurry may be continuously added to the feed tank together with some enzyme with a purge drawn from the recycle stream of equal volume.

In any event, running the process in a semi-continuous or continuous mode of operation permits permeation of solubilized components out of the reaction zone before they can be fur-ther broken down.

If the method of the invention is used for treating grounds from roast and ground coffee which has been previously extracted with water and/or thermally hydrolysed, the extract obtained from the method of this invention can be combined with the extracts obtained beforehand.

Where atmospheric grounds are used as the feed to the method of the invention, the extract produced may be combined with the extract obtained during the atmospheric extraction stage. The extracts are combined based on the ratio of extracted roasted yields from each stage. The combined extract is then concentrated, aromatised and dried as is conventional in the art.

The coffee extract can be dehydrated, such as a soluble coffee or dry mix composition, or it can be a ready-to-drink coffee product, a liquid mix composition, a frozen composition or a liquid concentrate composition. The coffee extract of the invention can also be used in non-beverage applications, such as instant desserts or confectionery products etc.

The processes to make those coffee compositions from soluble coffee extracts are known to a person skilled in the art.

In the method of the invention, water and enzyme is added to the coffee beans which may have been subjected to one or more first extractions.

Water may, e.g., be added so that the final concentration of dry matter is between 2%-30% (w/w), preferably between 5%-20% (w/w), such as about 10% (w/w).

The enzyme having mannanase activity may be added at a concentration of at least 0.001 g enzyme protein/kg dry matter, preferably at least 0.005 g enzyme protein/kg dry matter, such as at a concentration of 0.001-1 g enzyme protein/kg dry matter, preferably 0.005-0.5 g enzyme protein/kg dry matter.

The enzyme having mannanase activity may be added at a concentration of at least 0.001 g enzyme protein/kg coffee beans, preferably at least 0.005 g enzyme protein/kg coffee beans, such as at a concentration of 0.001-0.5 g enzyme protein/kg coffee beans, preferably 0.005-0.2 g enzyme protein/kg coffee beans.

The enzyme having mannanase activity is preferably added as an enzymatic preparation characterized in that at least 5%, preferably at least 10% or at least 20%, of the total protein in the preparation is an enzyme having mannanase activity as its predominant enzymatic activity.

The enzyme having mannanase activity may be added as a mixture with other enzymes such as, e.g., cellulase and/or galactanase enzyme(s).

In the method of the invention, after the water and the enzyme has been added to the roast and ground coffee beans which have optionally been subjected to one or more first extractions, the composition comprising the coffee beans, the water and the enzyme is incubated to make an aqueous coffee extract.

The incubation is to be performed at a temperature where the enzyme is active, typically in the range of about 25° C. to about 100° C. In the aspects of the invention where a thermostable enzyme is used, incubation may be performed at a temperature typically in the range of about 50° C. to about 100° C., preferably about 60° C. to about 100° C., more preferably about 70° C. to about 100° C., even more preferably about 80° C. to about 100° C.

The incubation may be performed for about 1 to about 48 hours, preferably about 2 to about 24 hours or about 4 to about 24 hours to permit enzyme reaction.

After the incubation, the coffee extract is separated from the extracted coffee beans by any means known in the art.

In one embodiment, the roast and ground coffee beans are subjected to one or more first extractions before step c, said one or more first extractions being denoted as step b, and a steam explosion is performed after step b and before step c. Alternatively, if more than one first extractions are performed, the steam explosion may be performed in between some of the first extractions and before step c.

In one embodiment, the roast and ground coffee beans are subjected to one or more first extractions before step c, said one or more first extractions being denoted as step b, and a second milling of the coffee beans is performed after step b and before step c. Alternatively, if more than one first extractions are performed, the second milling may be performed in between some of the first extractions and before step c.

In one embodiment, the roast and ground coffee beans are subjected to one or more first extractions before step c, said first extraction(s) being denoted as step b, and at least 8% by weight, preferably at least 10% by weight, of the dry matter of the partially extracted coffee beans obtained after step b is recovered in the coffee extract obtained in step e. In another embodiment, the roast and ground coffee beans are subjected to one or more first extractions before step c, said first extraction(s) being denoted as step b, and at least 12% by weight, preferably at least 14% by weight, of the dry matter of the partially extracted coffee beans obtained after step b is recovered in the coffee extract obtained in step e. In another embodiment, the roast and ground coffee beans are subjected to one or more first extractions before step c, said first extraction(s) being denoted as step b, and 8-40% by weight, preferably 10-30% or 12-25% by weight, of the dry matter of the partially extracted coffee beans obtained after step b is recovered in the coffee extract obtained in step e.

In one embodiment, the roast and ground coffee beans are subjected to one or more first extractions before step c and the coffee extract obtained in step e comprises at least 100% more dry matter, preferably at least 200% or at least 300% more dry matter, than a coffee extract prepared by a similar method without the addition of an enzyme having mannanase activity.

In some applications, the content of free monosaccharides in the coffee extract is important, since these may influence the taste of the coffee extract.

In one embodiment, the roast and ground coffee beans are subjected to one or more first extractions before step c and the coffee extract obtained in step e comprises at least 2% by weight, e.g. at least 5% or at least 8% by weight, of free monosaccharides based on the weight of the total sugars as monosaccharides. In another embodiment, the roast and ground coffee beans are subjected to one or more first extractions before step c and the coffee extract obtained in step e comprises 2-30% by weight, e.g. 5-30% or 8-30% by weight, of free monosaccharides based on the weight of the total sugars as monosaccharides.

In one embodiment, the roast and ground coffee beans are subjected to one or more first extractions before step c and the coffee extract obtained in step e comprises at least 2% by weight of free mannose based on the total weight of soluble coffee solids. In another embodiment, the roast and ground coffee beans are subjected to one or more first extractions before step c and the coffee extract obtained in step e comprises 2-5% by weight of free mannose based on the total weight of soluble coffee solids.

In one embodiment, the roast and ground coffee beans are subjected to one or more first extractions before step c and the free mannose content in the coffee extract obtained in step e is at least 5% by weight, preferably at least 10% by weight, of the total mannose content in said coffee extract. In another embodiment, the roast and ground coffee beans are subjected to one or more first extractions before step c and the free mannose content in the coffee extract obtained in step e is 5-30% by weight, preferably 10-25% by weight, of the total mannose content in said coffee extract.

Total mannose in the coffee extract in the context of the present invention means solubilized free mannose plus mannose bound in solubilized oligosaccharides.

Little or no content of glucose in coffee extracts is a quality parameter, and the glucose content has to be below 2.46% by weight to comply with the Commercial Item Description (CID) of May 16, 2013, authorized by the U.S. Department of Agriculture (USDA) (http://www.ams.usda.gov/AMSv1.0/getfile?dDocName=STELPRD3237484).

In one embodiment, the coffee extract obtained in step e comprises below 2.46% by weight, preferably below 2% by weight, more preferably below 1% or below 0.5% by weight, of total glucose based on the total weight of soluble coffee solids.

In one embodiment, the coffee extract obtained in step e comprises at least 15% by weight of total mannose based on the total weight of soluble coffee solids. In another embodiment, the coffee extract comprises 15-30% by weight of total mannose based on the total weight of soluble coffee solids.

### Polypeptides Having Endo-beta-1,4-mannanase Activity

In another aspect, the invention relates to polypeptides having endo-beta-1,4-mannanase activity and polynucleotides encoding the polypeptides. The invention also relates to nucleic acid constructs, vectors, and host cells comprising the polynucleotides as well as methods of producing and using the polypeptides.

In an embodiment, the present invention relates to polypeptides having a sequence identity to the polypeptide of SEQ ID NO: 3 of at least 75%, e.g., at least 80%, at least 85%, at least 90%, at least 91%, at least 92%, at least 93%, at least 94%, at least 95%, at least 96%, at least 97%, at least 98%, at least 99%, or 100%, which have endo-beta-1,4-mannanase activity. In one aspect, the polypeptides differ by up to 10 amino acids, e.g., 1, 2, 3, 4, 5, 6, 7, 8, 9, or 10, from the polypeptide of SEQ ID NO: 3.

In an embodiment, the present invention relates to polypeptides having a sequence identity to the polypeptide of SEQ ID NO: 8 of at least 90%, e.g., at least 91%, at least 92%, at least 93%, at least 94%, at least 95%, at least 96%, at least 97%, at least 98%, at least 99%, or 100%, which have endo-beta-1,4-mannanase activity. In one aspect, the polypeptides differ by up to 10 amino acids, e.g., 1, 2, 3, 4, 5, 6, 7, 8, 9, or 10, from the polypeptide of SEQ ID NO: 8.

In an embodiment, the present invention relates to polypeptides having a sequence identity to the polypeptide of SEQ ID NO: 13 of at least 80%, e.g., at least 85%, at least 90%, at least 91%, at least 92%, at least 93%, at least 94%, at least 95%, at least 96%, at least 97%, at least 98%, at least 99%, or 100%, which have endo-beta-1,4-mannanase activity. In one aspect, the polypeptides differ by up to 10 amino acids, e.g., 1, 2, 3, 4, 5, 6, 7, 8, 9, or 10, from the polypeptide of SEQ ID NO: 13.

In an embodiment, the polypeptide has been isolated.

In one embodiment, a polypeptide of the present invention preferably comprises or consists of the amino acid sequence of SEQ ID NO: 3 or an allelic variant thereof; or is a fragment thereof having endo-beta-1,4-mannanase activity. In another aspect, the polypeptide comprises or consists of the amino acid sequence of SEQ ID NO: 3.

In one embodiment, a polypeptide of the present invention preferably comprises or consists of the amino acid sequence of SEQ ID NO: 8 or an allelic variant thereof; or is a fragment thereof having endo-beta-1,4-mannanase activity. In another aspect, the polypeptide comprises or consists of the amino acid sequence of SEQ ID NO: 8.

In one embodiment, a polypeptide of the present invention preferably comprises or consists of the amino acid sequence of SEQ ID NO: 13 or an allelic variant thereof; or is a fragment thereof having endo-beta-1,4-mannanase activity. In another aspect, the polypeptide comprises or consists of the amino acid sequence of SEQ ID NO: 13.

In another embodiment, the present invention relates to a polypeptide having endo-beta-1,4-mannanase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii) the cDNA sequence thereof], or (iii) the full-length complement of (i) or (ii) (Sambrook et al., 1989, Molecular Cloning, A Laboratory Manual, 2d edition, Cold Spring Harbor, New York).

In another embodiment, the present invention relates to a polypeptide having endo-beta-1,4-mannanase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 6, (ii) the cDNA sequence thereof], or (iii) the full-length complement of (i) or (ii).

In another embodiment, the present invention relates to a polypeptide having endo-beta-1,4-mannanase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 11, (ii) the cDNA sequence thereof], or (iii) the full-length complement of (i) or (ii).

The polynucleotide of any of SEQ ID NO: 1, 6 or 11 or a subsequence of any of these, as well as the polypeptide of SEQ ID NO: 2, 7 or 12 or a fragment of any of these, may be used to design nucleic acid probes to identify and clone DNA encoding polypeptides having endo-beta-1,4-mannanase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having endo-beta-1,4-mannanase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

For purposes of the present invention, hybridization indicates that the polynucleotide hybridizes to a labeled nucleic acid probe corresponding to (i) SEQ ID NO: 1, 6 or 11; (ii) the mature polypeptide coding sequence of SEQ ID NO: 1, 6 or 11; (iii) the cDNA sequence thereof]; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In another embodiment, the present invention relates to a polypeptide having endo-beta-1,4-mannanase activity encoded by a polynucleotide having a sequence identity to the mature polypeptide coding sequence of SEQ ID NO: 1 or the cDNA sequence thereof of at least 75%, e.g., at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to a polypeptide having endo-beta-1,4-mannanase activity encoded by a polynucleotide having a sequence identity to the mature polypeptide coding sequence of SEQ ID NO: 6 or the cDNA sequence thereof of at least 90%, e.g., at least 91%, at least 92%, at least 93%, at least 94%, at least 95%, at least 96%, at least 97%, at least 98%, at least 99%, or 100%.

In another embodiment, the present invention relates to a polypeptide having endo-beta-1,4-mannanase activity encoded by a polynucleotide having a sequence identity to the mature polypeptide coding sequence of SEQ ID NO: 11 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%.

In another embodiment, the present invention relates to variants of the polypeptide of any of SEQ ID NO: 3, 8 or 13 comprising a substitution, deletion, and/or insertion at one or more (e.g., several) positions. In an embodiment, the number of amino acid substitutions, deletions and/or insertions introduced into the polypeptide of any of SEQ ID NO: 3, 8 or 13 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino-or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/lle, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/lle, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for endo-beta-1,4-mannanase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992 FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Endo-beta-1,4-mannanase Activity

A polypeptide having endo-beta-1,4-mannanase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

In one aspect, the polypeptide is a Talaromyces polypeptide, e.g., a polypeptide obtained from Talaromyces leycettanus.

In another aspect, the polypeptide is a Chaetomium polypeptide, e.g., a polypeptide obtained from Chaetomium virescens.

In another aspect, the polypeptide is a Sordaria polypeptide, e.g., a polypeptide obtained from Sordaria macrospora.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Catalytic Domains

In one embodiment, the present invention also relates to catalytic domains having a sequence identity to amino acids 75 to 414 of SEQ ID NO: 3 of at least 75%, e.g., at least 80%, at least 85%, at least 90%, at least 95%, at least 96%, at least 97%, at least 98%, at least 99%, or 100%. In one aspect, the catalytic domains comprise amino acid sequences that differ by up to 10 amino acids, e.g., 1, 2, 3, 4, 5, 6, 7, 8, 9, or 10, from amino acids 75 to 414 of SEQ ID NO: 3.

The catalytic domain preferably comprises or consists of amino acids 75 to 414 of SEQ ID NO: 3 or an allelic variant thereof; or is a fragment thereof having endo-beta-1,4-mannanase activity.

In another embodiment, the present invention also relates to catalytic domains encoded by polynucleotides that hybridize under very low stringency conditions, low stringency conditions, medium stringency conditions, medium-high stringency conditions, high stringency conditions, or very high stringency conditions (as defined above) with (i) the nucleotides 317 to 1545 of SEQ ID NO: 1, (ii) the cDNA sequence thereof, or (iii) the full-length complement of (i) or (ii) (Sambrook et al., 1989, supra).

In another embodiment, the present invention also relates to catalytic domains encoded by polynucleotides having a sequence identity to nucleotides 317 to 1545 of SEQ ID NO: 1 or the cDNA sequence thereof of at least 75%, e.g., at least 80%, at least 85%, at least 90%, at least 95%, at least 96%, at least 97%, at least 98%, at least 99%, or 100%.

The polynucleotide encoding the catalytic domain preferably comprises or consists of nucleotides 317 to 1545 of SEQ ID NO: 1.

In another embodiment, the present invention also relates to catalytic domain variants of amino acids 75 to 414 of SEQ ID NO: 3 comprising a substitution, deletion, and/or insertion at one or more (e.g., several) positions. In one aspect, the number of amino acid substitutions, deletions and/or insertions introduced into the sequence of amino acids 75 to 414 of SEQ ID NO: 3 is up to 10, e.g., 1, 2, 3, 4, 5, 6, 8, 9, or 10.

### Binding Domains

In one embodiment, the present invention also relates to a CBM1 binding domains having a sequence identity to amino acids 1 to 37 of SEQ ID NO: 3 of at least 75%, e.g., at least 80%, at least 85%, at least 90%, at least 91%, at least 92%, at least 93%, at least 94%, at least 95%, at least 96%, at least 97%, at least 98%, at least 99%, or 100%. In one aspect, the CBM1 binding domains comprise amino acid sequences that differ by up to 10 amino acids, e.g., 1, 2, 3, 4, 5, 6, 7, 8, 9, or 10, from amino acids 1 to 37 of SEQ ID NO: 3.

The CBM1 binding domain preferably comprises or consists of amino acids 1 to 37 of SEQ ID NO: 3 or an allelic variant thereof; or is a fragment thereof having CBM1 binding activity.

In another embodiment, the present invention also relates to CBM1 binding domains encoded by polynucleotides that hybridize under very low stringency conditions, low stringency conditions, medium stringency conditions, medium-high stringency conditions, high stringency conditions, or very high stringency conditions (as defined above) with (i) the nucleotides 1 to 111 of SEQ ID NO: 1, (ii) the cDNA sequence thereof, or (iii) the full-length complement of (i) or (ii) (Sambrook et al., 1989, supra).

In another embodiment, the present invention also relates to CBM1 binding domains encoded by polynucleotides having a sequence identity to nucleotides 1 to 111 of SEQ ID NO: 1 of at least 75%, e.g., at least 80%, at least 85%, at least 90%, at least 91%, at least 92%, at least 93%, at least 94%, at least 95%, at least 96%, at least 97%, at least 98%, at least 99%, or 100%.

The polynucleotide encoding the CBM1 binding domain preferably comprises or consists of nucleotides 1 to 111 of SEQ ID NO: 1.

In another embodiment, the present invention also relates to CBM1 binding domain variants of amino acids 1 to 37 of SEQ ID NO: 3 comprising a substitution, deletion, and/or insertion at one or more (e.g., several) positions. In one aspect, the number of amino acid substitutions, deletions and/or insertions introduced into the sequence of amino acids 1 to 37 of SEQ ID NO: 3 is up to 10, e.g., 1, 2, 3, 4, 5, 6, 8, 9, or 10.

A catalytic domain operably linked to the CBM1 binding domain may be from a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase. The polynucleotide encoding the catalytic domain may be obtained from any prokaryotic, eukaryotic, or other source.

### Polynucleotides

The present invention also relates to polynucleotides encoding a polypeptide, a catalytic domain, or CBM1 binding domain of the present invention, as described herein. In an embodiment, the polynucleotide encoding the polypeptide, catalytic domain, or CBM1 binding domain of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Talaromyces, Chaetomium, or Sordaria, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, 6 or 11, or the cDNA sequence thereof, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488. The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergilluscell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome. For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980). The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolismmay be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a polypeptide of the present invention. Preferably, the compositions are enriched in such a polypeptide. The term “enriched” indicates that the endo-beta-1,4-mannanase activity of the composition has been increased, e.g., with an enrichment factor of at least 1.1.

The compositions may comprise a polypeptide of the present invention as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

### Uses

The present invention also relates to use of a polypeptide of the invention in coffee extraction. The present invention also relates to a method for producing a coffee extract, comprising the steps:
a. providing roast and ground coffee beans;
b. adding to said coffee beans water and a polypeptide of the invention having endo-beta-1,4-mannanase activity;
c. incubating to make an aqueous coffee extract; and
d. separating the coffee extract from the extracted coffee beans.

### Embodiments

The invention is further defined in the following paragraphs:

1. A method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
   wherein the enzyme having mannanase activity is thermostable.

2. A method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
   wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to any of SEQ ID NO: 3, SEQ ID NO: 8, SEQ ID NO: 13 or SEQ ID NO: 18.

3. The method of paragraph 2, wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to SEQ ID NO: 3 or SEQ ID NO: 18.

4. The method of paragraph 3, wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to SEQ ID NO: 3.

5. The method of paragraph 3, wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to SEQ ID NO: 18.

6. The method of any of paragraphs 3-5, wherein the enzyme having mannanase activity is thermostable.

7. The method of any of paragraphs 1 or 6, wherein the enzyme having mannanase activity has a melting temperature (Tₘ) determined by Differential Scanning Calorimetry (DSC) of at least 80° C., preferably at least 85° C. or at least 90° C.

8. The method of any of paragraphs 1 or 6-7, wherein the incubation in step d. is performed at a temperature of at least 60° C. such as at least 65° C., preferably at least 70° C. such as at least 75° C. or at least 80° C.

9. The method of any of paragraphs 1 or 6-8, wherein the incubation in step d is performed for at least one hour, preferably for at least 2 hours or at least 4 hours.

10. The method of any of paragraphs 1 or 6-9, wherein the incubation in step d is performed for 1-48 hours, preferably for 2-24 hours or 4-24 hours.

11. The method of paragraph 2, wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to any of SEQ ID NO: 8 or SEQ ID NO: 13.

12. The method of any of the preceding paragraphs, wherein the enzyme having mannanase activity is an endo-beta-1,4-mannanase.

13. The method of any of the preceding paragraphs, wherein the enzyme having mannanase activity is a GH5 endo-beta-1,4-mannanase, preferably a GH5_7 endo-beta-1,4-mannanase or a GH5_8 endo-beta-1,4-mannanase.

14. The method of any of the preceding paragraphs, wherein the roast and ground coffee beans are subjected to a first extraction before step c.

15. The method of any of the preceding paragraphs, wherein the roast and ground coffee beans are subjected to one or more extractions before step c.

16. The method of any of paragraphs 14-15, wherein a steam explosion is performed after the one or more first extractions (step b) and before step c.

17. The method of any of paragraphs 14-16, wherein a second milling of the coffee beans is performed after the one or more first extractions (step b) and before step c.

18. The method of any of the preceding paragraphs, wherein the coffee extract obtained in step e comprises at least 100% more dry matter than a coffee extract prepared by a similar method without the addition of an enzyme having mannanase activity.

19. The method of any of paragraphs 14-18, wherein at least 8% by weight of the dry matter of the partially extracted coffee beans obtained after step b is recovered in the coffee extract obtained in step e.

20. A polypeptide having endo-beta-1,4-mannanase activity, selected from the group consisting of:
(a) a polypeptide having at least 90% sequence identity to the polypeptide of SEQ ID NO: 8; and
(b) a polypeptide having at least 80% sequence identity to the polypeptide of SEQ ID NO: 13.

21. The polypeptide of paragraph 20, which is a GH5_7 endo-beta-1,4-mannanase.

22. The polypeptide of paragraph 20 or 21, selected from the group consisting of:
(a) a polypeptide having at least 91%, at least 92%, at least 93%, at least 94%, at least 95%, at least 96%, at least 97%, at least 98%, at least 99% or 100% sequence identity to the polypeptide of SEQ ID NO: 8; and
(b) a polypeptide having at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 13.

23. The polypeptide of paragraph 20 or 21, selected from the group consisting of
(a) a polypeptide differing by up to 10 amino acids, e.g., 1, 2, 3, 4, 5, 6, 7, 8, 9, or 10, from the polypeptide of SEQ ID NO: 8; and
(b) a polypeptide differing by up to 10 amino acids, e.g., 1, 2, 3, 4, 5, 6, 7, 8, 9, or 10, from the polypeptide of SEQ ID NO: 13.

24. The polypeptide of paragraph 20 or 21, selected from the group consisting of
(a) a polypeptide differing by up to 5 amino acids, e.g., 1, 2, 3, 4 or 5, from the polypeptide of SEQ ID NO: 8; and
(b) a polypeptide differing by up to 5 amino acids, e.g., 1, 2, 3, 4 or 5, from the polypeptide of SEQ ID NO: 13.

25. The polypeptide of paragraph 20, comprising or consisting of:
(a) the polypeptide of SEQ ID NO: 8; or
(b) the polypeptide of SEQ ID NO: 13.

262 The polypeptide of any of paragraphs 20-25, which is an isolated polypeptide.

27. A composition comprising the polypeptide of any of paragraphs 20-26.

282 Use of the polypeptide of any of paragraphs 20-26 for treatment of coffee.

29. Use of the polypeptide of any of paragraphs 20-26 for treatment of roast and ground coffee beans.

30. The use of paragraph 29, wherein the roast and ground coffee beans have been partially extracted.

31. A method for producing a coffee extract, comprising the steps:
a. providing roast and ground coffee beans;
b. adding to said coffee beans water and the polypeptide of any of paragraphs 20-26;
c. incubating to make an aqueous coffee extract; and
d. separating the coffee extract from the extracted coffee beans.

32. The method of paragraph 31, wherein the roast and ground coffee beans have been partially extracted.

33. An isolated polynucleotide encoding the polypeptide of any of paragraphs 20-26.

34. A nucleic acid construct or expression vector comprising the polynucleotide of paragraph 33 operably linked to one or more control sequences that direct the production of the polypeptide in an expression host.

35. A recombinant host cell comprising the polynucleotide of paragraph 33 operably linked to one or more control sequences that direct the production of the polypeptide.

36. A method of producing the polypeptide of any of paragraphs 20-26, comprising cultivating a cell, which in its wild-type form produces the polypeptide, under conditions conducive for production of the polypeptide.

37. The method of paragraph 36, further comprising recovering the polypeptide.

38. A method of producing a polypeptide having endo-beta-1,4-mannanase activity, comprising cultivating the host cell of paragraph 35 under conditions conducive for production of the polypeptide.

39. The method of paragraph 38, further comprising recovering the polypeptide.

40. A whole broth formulation or cell culture composition comprising a polypeptide of any of paragraphs 20-26.

41. A polypeptide having endo-beta-1,4-mannanase activity, selected from the group consisting of:
(a) a polypeptide having at least 90% sequence identity to the polypeptide of SEQ ID NO: 8;
(b) a polypeptide encoded by a polynucleotide that hybridizes under low stringency conditions with (i) the mature polypeptide coding sequence of SEQ ID NO: 6, (ii) the cDNA sequence thereof, or (iii) the full-length complement of (i) or (ii);
(c) a polypeptide encoded by a polynucleotide having at least 90% sequence identity to the mature polypeptide coding sequence of SEQ ID NO: 6 or the cDNA sequence thereof;
(d) a variant of the polypeptide of SEQ ID NO: 8 comprising a substitution, deletion, and/or insertion at one or more positions; and
(e) a fragment of the polypeptide of (a), (b), (c), or (d) that has endo-beta-1,4-mannanase activity.

42. The polypeptide of paragraph 41, which is a GH5_7 endo-beta-1,4-mannanase.

43. The polypeptide of paragraph 41 or 42, having at least 90%, at least 91%, at least 92%, at least 93%, at least 94%, at least 95%, at least 96%, at least 97%, at least 98%, at least 99% or 100% sequence identity to the polypeptide of SEQ ID NO: 8.

44. The polypeptide of any of paragraphs 41-43 differing by up to 10 amino acids, e.g., 1, 2, 3, 4, 5, 6, 7, 8, 9, or 10, from the polypeptide of SEQ ID NO: 8.

45. The polypeptide of any of paragraphs 41-44, which is encoded by a polynucleotide that hybridizes under low stringency conditions, low-medium stringency conditions, medium stringency conditions, medium-high stringency conditions, high stringency conditions, or very high stringency conditions with (i) the mature polypeptide coding sequence of SEQ ID NO: 6, (ii) the cDNA sequence thereof, or (iii) the full-length complement of (i) or (ii).

46. The polypeptide of any of paragraphs 41-45, which is encoded by a polynucleotide having at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 6 or the cDNA sequence thereof.

47. The polypeptide of any of paragraphs 41-46, comprising or consisting of the polypeptide of SEQ ID NO: 8.

48. The polypeptide of any of paragraphs 41-46, which is a variant of the polypeptide of SEQ ID NO: 8 comprising a substitution, deletion, and/or insertion at one or more positions.

49. The polypeptide of any of paragraphs 41-46, which is a fragment of SEQ ID NO: 8, wherein the fragment has endo-beta-1,4-mannanase activity.

50. The polypeptide of any of paragraphs 41-49, which is obtained from Chaetomium.

51. The polypeptide of paragraph 50, which is obtained from Chaetomium virescens.

52. The polypeptide of any of paragraphs 41-51, which is an isolated polypeptide.

53. A composition comprising the polypeptide of any of paragraphs 41-51.

54. Use of the polypeptide of any of paragraphs 41-51 for treatment of coffee.

55. Use of the polypeptide of any of paragraphs 41-51 for treatment of roast and ground coffee beans.

56. The use of paragraph 55, wherein the roast and ground coffee beans have been partially extracted.

57. A method for producing a coffee extract, comprising the steps:
a. providing roast and ground coffee beans;
b. adding to said coffee beans water and the polypeptide of any of paragraphs 41-51;
c. incubating to make an aqueous coffee extract; and
d. separating the coffee extract from the extracted coffee beans.

58. The method of paragraph 57, wherein the roast and ground coffee beans have been partially extracted.

59. An isolated polynucleotide encoding the polypeptide of any of paragraphs 41-51.

60. A nucleic acid construct or expression vector comprising the polynucleotide of paragraph 59 operably linked to one or more control sequences that direct the production of the polypeptide in an expression host.

61. A recombinant host cell comprising the polynucleotide of paragraph 59 operably linked to one or more control sequences that direct the production of the polypeptide.

62. A method of producing the polypeptide of any of paragraphs 41-51, comprising cultivating a cell, which in its wild-type form produces the polypeptide, under conditions conducive for production of the polypeptide.

63. The method of paragraph 62, further comprising recovering the polypeptide.

64. A method of producing a polypeptide having endo-beta-1,4-mannanase activity, comprising cultivating the host cell of paragraph 61 under conditions conducive for production of the polypeptide.

65. The method of paragraph 64, further comprising recovering the polypeptide.

66. An isolated polynucleotide encoding a signal peptide comprising or consisting of amino acids 1 to 17 of SEQ ID NO: 7.

67. A whole broth formulation or cell culture composition comprising a polypeptide of any of paragraphs 41-51.

68. A polypeptide having endo-beta-1,4-mannanase activity, selected from the group consisting of:
(a) a polypeptide having at least 80% sequence identity to the polypeptide of SEQ ID NO: 13;
(b) a polypeptide encoded by a polynucleotide that hybridizes under low stringency conditions with (i) the mature polypeptide coding sequence of SEQ ID NO: 11, (ii) the cDNA sequence thereof, or (iii) the full-length complement of (i) or (ii);
(c) a polypeptide encoded by a polynucleotide having at least 80% sequence identity to the mature polypeptide coding sequence of SEQ ID NO: 11 or the cDNA sequence thereof;
(d) a variant of the polypeptide of SEQ ID NO: 13 comprising a substitution, deletion, and/or insertion at one or more positions; and
(e) a fragment of the polypeptide of (a), (b), (c), or (d) that has endo-beta-1,4-mannanase activity.

69. The polypeptide of paragraph 68, which is a GH5_7 endo-beta-1,4-mannanase.

70. The polypeptide of paragraph 68 or 69, having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 13.

71. The polypeptide of any of paragraphs 68-70 differing by up to 10 amino acids, e.g., 1, 2, 3, 4, 5, 6, 7, 8, 9, or 10, from the polypeptide of SEQ ID NO: 13.

72. The polypeptide of any of paragraphs 68-71, which is encoded by a polynucleotide that hybridizes under low stringency conditions, low-medium stringency conditions, medium stringency conditions, medium-high stringency conditions, high stringency conditions, or very high stringency conditions with (i) the mature polypeptide coding sequence of SEQ ID NO: 11, (ii) the cDNA sequence thereof, or (iii) the full-length complement of (i) or (ii).

73. The polypeptide of any of paragraphs 68-72, which is encoded by a polynucleotide having at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 11 or the cDNA sequence thereof.

74. The polypeptide of any of paragraphs 68-73, comprising or consisting of the polypeptide of SEQ ID NO: 13.

75. The polypeptide of any of paragraphs 68-73, which is a variant of the polypeptide of SEQ ID NO: 13 comprising a substitution, deletion, and/or insertion at one or more positions.

76. The polypeptide of any of paragraphs 68-74, which is a fragment of SEQ ID NO: 13, wherein the fragment has endo-beta-1,4-mannanase activity.

77. The polypeptide of any of paragraphs 68-76, which is obtained from Sordaria.

78. The polypeptide of paragraph 77, which is obtained from Sordaria macrospora.

79. The polypeptide of any of paragraphs 68-78, which is an isolated polypeptide.

80. A composition comprising the polypeptide of any of paragraphs 68-78.

81. Use of the polypeptide of any of paragraphs 68-78 for treatment of coffee.

82. Use of the polypeptide of any of paragraphs 68-78 for treatment of roast and ground coffee beans.

83. The use of paragraph 82, wherein the roast and ground coffee beans have been partially extracted.

84 A method for producing a coffee extract, comprising the steps:
a. providing roast and ground coffee beans;
b. adding to said coffee beans water and the polypeptide of any of paragraphs 68-78;
c. incubating to make an aqueous coffee extract; and
d. separating the coffee extract from the extracted coffee beans.

85. The method of paragraph 84, wherein the roast and ground coffee beans have been partially extracted.

86. An isolated polynucleotide encoding the polypeptide of any of paragraphs 68-78.

87. A nucleic acid construct or expression vector comprising the polynucleotide of paragraph 86 operably linked to one or more control sequences that direct the production of the polypeptide in an expression host.

88. A recombinant host cell comprising the polynucleotide of paragraph 86 operably linked to one or more control sequences that direct the production of the polypeptide.

89. A method of producing the polypeptide of any of paragraphs 68-78, comprising cultivating a cell, which in its wild-type form produces the polypeptide, under conditions conducive for production of the polypeptide.

90. The method of paragraph 89, further comprising recovering the polypeptide.

91. A method of producing a polypeptide having endo-beta-1,4-mannanase activity, comprising cultivating the host cell of paragraph 88 under conditions conducive for production of the polypeptide.

92. The method of paragraph 91, further comprising recovering the polypeptide.

93. An isolated polynucleotide encoding a signal peptide comprising or consisting of amino acids 1 to 17 of SEQ ID NO: 12.

94. A whole broth formulation or cell culture composition comprising a polypeptide of any of paragraphs 68-78.

95. The method of any of paragraphs 1-19 wherein the enzyme having mannanase activity comprises a CBM1 binding domain.

96. A method for producing a coffee extract, comprising the steps:
a. providing roast and ground coffee beans;
b. optionally performing one or more first extractions of said coffee beans;
c. adding to said coffee beans, which have optionally been subjected to one or more first extractions, water and an enzyme having mannanase activity;
d. incubating to make an aqueous coffee extract; and
e. separating the coffee extract from the extracted coffee beans,
   wherein the enzyme having mannanase activity comprises a CBM1 binding domain.

97. The method of paragraph 96 wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to any of SEQ ID NO: 3, SEQ ID NO: 8, SEQ ID NO: 13; SEQ ID NO: 18 or SEQ ID NO: 19.

98. The method of any of paragraphs 96-97, wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to SEQ ID NO: 3 or SEQ ID NO: 19.

99. The method of any of paragraphs 96-98, wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to SEQ ID NO: 3.

100. The method of any of paragraphs 96-98, wherein the enzyme having mannanase activity has at least 60% sequence identity, preferably at least 65%, at least 70%, at least 75%, at least 80%, at least 85%, at least 90%, at least 95%, at least 96%, at least 97%, at least 98%, at least 99% or 100% sequence identity, to SEQ ID NO: 19.

101. The method of any of paragraphs 96-100, wherein the enzyme having mannanase activity is thermostable.

102. The method of any of paragraphs 96-101, wherein the enzyme having mannanase activity has a melting temperature (Tₘ) determined by Differential Scanning Calorimetry (DSC) of at least 80° C., preferably at least 85° C. or at least 90° C.

103. The method of any of paragraphs 96-102, wherein the incubation in step d. is performed at a temperature of at least 60° C. such as at least 65° C., preferably at least 70° C. such as at least 75° C. or at least 80° C.

104. The method of any of paragraphs 96-103, wherein the incubation in step d is performed for at least one hour, preferably for at least 2 hours or at least 4 hours.

105. The method of any of paragraphs 96-104, wherein the incubation in step d is performed for 1-48 hours, preferably for 2-24 hours or 4-24 hours.

106. The method of any of paragraphs 96-105, wherein the enzyme having mannanase activity is an endo-beta-1,4-mannanase.

107. The method of any of paragraphs 96-106, wherein the enzyme having mannanase activity is a GH5 endo-beta-1,4-mannanase, preferably a GH5_7 endo-beta-1,4-mannanase or a GH5_8 endo-beta-1,4-mannanase.

108. The method of any of paragraphs 96-107, wherein the roast and ground coffee beans are subjected to a first extraction before step c.

109. The method of any of paragraphs 96-108, wherein the roast and ground coffee beans are subjected to one or more extractions before step c.

110. The method of any of paragraphs 108-109, wherein a steam explosion is performed after the one or more first extractions (step b) and before step c.

111. The method of any of paragraphs 108-110, wherein a second milling of the coffee beans is performed after the one or more first extractions (step b) and before step c.

112. The method of any of paragraphs 96-111, wherein the coffee extract obtained in step e comprises at least 100% more dry matter than a coffee extract prepared by a similar method without the addition of an enzyme having mannanase activity.

113. The method of any of paragraphs 96-112, wherein at least 8% by weight of the dry matter of the partially extracted coffee beans obtained after step b is recovered in the coffee extract obtained in step e.

### EXAMPLES

### Enzymes

In the examples below the following enzymes were used:

| SEQ | | | | |
|---|---|---|---|---|
| ID | | | | |
| NO: | Example | Description | Origin | GH |
| 1-3 | Example 1 | Endo-β-1,4-mannanase | Talaromyces | GH5 |
| | | | leycettanus | |
| 6-8 | Example 2 | Endo-β-1,4-mannanase | Chaetomium virescens | GH5 |
| 11-13 | Example 3 | Endo-β-1,4-mannanase | Sordaria macrospora | GH5 |

### Materials

Chemicals used as buffers and substrates were commercial products of at least reagent grade.

### Media and Solutions

YP+2% glucose medium was composed of 1% yeast extract, 2% peptone and 2% glucose.

PDA agar plates were composed of potato infusion (potato infusion was made by boiling 300 g of sliced (washed but unpeeled) potatoes in water for 30 minutes and then decanting or straining the broth through cheesecloth. Distilled water was then added until the total volume of the suspension was one liter, followed by 20 g of dextrose and 20 g of agar powder. The medium was sterilized by autoclaving at 15 psi for 15 minutes (Bacteriological Analytical Manual, 8th Edition, Revision A, 1998).

LB plates were composed of 10 g of Bacto-Tryptone, 5 g of yeast extract, 10 g of sodium chloride, 15 g of Bacto-agar, and deionized water to 1 liter. The medium was sterilized by autoclaving at 15 psi for 15 minutes (Bacteriological Analytical Manual, 8th Edition, Revision A, 1998).

COVE sucrose plates were composed of 342 g Sucrose (Sigma S-9378), 20 g Agar powder, 20 ml Cove salt solution (26 g MgSO₄·7H₂O, 26 g KCL, 26 g KH₂PO₄, 50 ml Cove trace metal solution) and deionized water to 1 liter), and deionized water to 1 liter). The medium was sterilized by autoclaving at 15 psi for 15 minutes (Bacteriological Analytical Manual, 8th Edition, Revision A, 1998). The medium was cooled to 60° C. and added 10 mM acetamide, 15 mM CsCl, Triton X-100 (50 μl/500 ml)).

Cove trace metal solution was composed of 0.04 g Na₂B₄O₇·10H₂O, 0.4 g CuSO₄·5H₂O, 1.2 g FeSO₄·7H₂O, 0.7 g MnSO₄·H₂O, 0.8 g Na₂MoO₄·2H₂O, 10 g ZnSO₄·7H₂O, and deionized water to 1 liter.

### Example 1: Cloning, Expression and Purification of the Talaromyces leycettanus Endo-Mannanase (Mannanase 1)

### Strains

Talaromyces leycettanus Strain CBS398.68 was used as the source of a polypeptide having mannanase activity. Aspergillus oryzae MT3568 strain was used for expression of the Talaromyces leycettanus gene encoding the polypeptide having mannanase activity. A. oryzae MT3568 is an amdS (acetamidase) disrupted gene derivative of Aspergillus oryzae JaL355 (WO 02/40694) in which pyrG auxotrophy was restored by disrupting the A. oryzae acetamidase (amdS) gene.

### Source of DNA Sequence Information for Talaromyces leycettanus Strain CBS398.68

Genomic sequence information was generated by Illumina DNA sequencing at the Beijing Genome Institute (BGI) in Beijing, China from genomic DNA isolated from Talaromyces leycettanus Strain CBS398.68. A preliminary assembly of the genome was analyzed using the Pedant-Pro™ Sequence Analysis Suite (Biomax Informatics AG, Martinsried, Germany). Gene models constructed by the software were used as a starting point for detecting GH5 homologues in the genome. More precise gene models were constructed manually using multiple known GH5 protein sequences as a guide.

### Talaromyces leycettanus Strain CBS398.68 Genomic DNA Extraction

To generate genomic DNA for PCR amplification, Talaromyces leycettanus Strain CBS398.68 was propagated on PDA agar plates by growing at 26° C. for 7 days. Spores harvested from the PDA plates were used to inoculate 25 ml of YP+2% glucose medium in a baffled shake flask and incubated at 26° C. for 72 hours with agitation at 85 rpm.

Genomic DNA was isolated according to a modified DNeasy Plant Maxi kit protocol (Qiagen Danmark, Copenhagen, Denmark). The fungal material from the above culture was harvested by centrifugation at 14,000×g for 2 minutes. The supernatant was removed and the 0.5 g of the pellet was frozen in liquid nitrogen with quartz sand and grinded to a fine powder in a pre-chilled mortar. The powder was transferred to a 15 ml centrifuge tube and added 5 ml buffer AP1 (preheated to 65° C.) and 10 μl RNase A stock solution (100 mg/ml) followed by vigorous vortexing. After incubation for 10 minutes at 65° C. with regular inverting of the tube, 1.8 ml buffer AP2 was added to the lysate by gentle mixing followed by incubation on ice for 10 min. The lysate was then centrifugated at 3000×g for 5 minutes at room temperature and the supernatant was decanted into a QIAshredder maxi spin column placed in a 50 ml collection tube. This was followed by centrifugation at 3000×g for 5 minutes at room temperature. The flow-through was transferred into a new 50 ml tube and added 1.5 volumes of buffer AP3/E followed by vortexing. 15 ml of the sample was transferred into a DNeasy Maxi spin column placed in a 50 ml collection tube and centrifuged at 3000×g for 5 minutes at room temperature. The flow-through was discarded and 12 ml buffer AW was added to the DNeasy Maxi spin column placed in a 50 ml collection tube and centrifuged at 3000×g for 10 minutes at room temperature. After discarding the flow-through, centrifugation was repeated to dispose of the remaining alcohol. The DNeasy Maxi spin column was transferred to a new 50 ml tube and 0.5 ml buffer AE (preheated to 70° C.) was added. After incubation for 5 minutes at room temperature, the sample was eluded by centrifugation at 3000×g for 5 minutes at room temperature. Elution was repeated with an additional 0.5 ml buffer AE and the eluates were combined. The concentration of the harvested DNA was measured by a UV spectrophotometer at 260 nm.

### Construction of an Aspergillus oryzae Expression Vector Containing Talaromyces leycettanus Strain CBS398.68 Genomic Sequence Encoding a Family GH5 Polypeptide Having Mannanase Activity

Two synthetic oligonucleotide primers shown below were designed to PCR amplify the Talaromyces leycettanus Strain CBS398.68 P23YST gene (SEQ ID NO: 1) from the genomic DNA prepared as described above. An IN-FUSION™ Cloning Kit (BD Biosciences, Palo Alto, CA, USA) was used to clone the fragment directly into the expression vector pDau109 (WO 2005/042735).

F-P23YST
(SEQ ID NO: 4)
5′-acacaactggggatccaccATGAAGTTGTCTACCCTCAATTTCCT-
3′
R-P23YST
(SEQ ID NO: 5)
5′-ccctctagatctcgagCACGTCAGTATCAGCGAAGCAT-3′

Capital letters represent gene sequence. The underlined sequence is homologous to the insertion sites of pDau109.

An MJ Research PTC-200 DNA engine was used to perform the PCR reaction. A Phusion® High-Fidelity PCR Kit (Finnzymes Oy, Espoo, Finland) was used for the PCR amplification. The PCR reaction was composed of 5 μl of 5×HF buffer (Finnzymes Oy, Espoo, Finland), 0.5 μl of dNTPs (10 mM), 0.5 μl of Phusion® DNA polymerase (0.2 units/μl) (Finnzymes Oy, Espoo, Finland), 2 μl of primer F-P23YST (2.5 M), 2 μl of primer R-P23YST (2.5 μM), 0.5 μl of Talaromyces leycettanus genomic DNA (100 ng/μl), and 14.5 μl of deionized water in a total volume of 25 μl. The PCR conditions were 1 cycle at 95° C. for 2 minutes. 35 cycles each at 98° C. for 10 seconds, 60° C. for 30 seconds, and 72° C. for 2 minutes; and 1 cycle at 72° C. for 10 minutes. The sample was then held at 12° C. until removed from the PCR machine.

The reaction products were isolated by 1.0% agarose gel electrophoresis using 40 mM Tris base, 20 mM sodium acetate, 1 mM disodium EDTA (TAE) buffer where a 1613 bp product band was excised from the gel and purified using an illustra GFX® PCR DNA and Gel Band Purification Kit (GE Healthcare Life Sciences, Brondby, Denmark) according to the manufacturer's instructions. The fragment was then cloned into Bam HI and Xho I digested pDau109 using an IN-FUSION™ Cloning Kit resulting in plasmid pP23YST. Cloning of the P23YST gene into Bam HI-Xho I digested pDau109 resulted in the transcription of the Talaromyces leycettanus P23YST gene under the control of a NA2-tpi double promoter. NA2-tpi is a modified promoter from the gene encoding the Aspergillus niger neutral alpha-amylase in which the untranslated leader has been replaced by an untranslated leader from the gene encoding the Aspergillus nidulans triose phosphate isomerase.

The cloning protocol was performed according to the IN-FUSION™ Cloning Kit instructions generating a P23YST GH5 construct. The treated plasmid and insert were transformed into One Shot® TOP10F′ Chemically Competent E. coli cells (Invitrogen, Carlsbad, CA, USA) according to the manufacturer's protocol and plated onto LB plates supplemented with 0.1 mg of ampicillin per ml. After incubating at 37° C. overnight, colonies were seen growing under selection on the LB ampicillin plates. Two colonies transformed with the P23YST GH5 construct were cultivated in LB medium supplemented with 0.1 mg of ampicillin per ml and plasmid was isolated with a QIAprep Spin Miniprep Kit (QIAGEN Inc., Valencia, CA, USA) according to the manufacturer's protocol.

Isolated plasmids were sequenced with vector primers and P23YST gene specific primers in order to determine a representative plasmid expression clone that was free of PCR errors.

### Characterization of the Talaromyces leycettanus CBS398.68 Genomic Sequence Encoding a P23YST GH5 Polypeptide Having Mannanase Activity

DNA sequencing of the Talaromyces leycettanus CBS398.68 P23YST GH5 genomic clone was performed with an Applied Biosystems Model 3700 Automated DNA Sequencer using version 3.1 BIG-DYE™ terminator chemistry (Applied Biosystems, Inc., Foster City, CA, USA) and primer walking strategy. Nucleotide sequence data were scrutinized for quality and all sequences were compared to each other with assistance of PHRED/PHRAP software (University of Washington, Seattle, WA, USA).

The nucleotide sequence and deduced amino acid sequence of the Talaromyces leycettanus P23YST gene is shown in SEQ ID NO: 1 and SEQ ID NO: 2, respectively. The coding sequence is 1548 bp including the stop codon and is interrupted by three introns. The encoded predicted protein is 431 amino acids. Using the SignalP program (Nielsen et al., 1997, Protein Engineering 10: 1-6), a signal peptide of 17 residues was predicted. The predicted mature protein (SEQ ID NO: 3) contains 414 amino acids with a predicted molecular mass of 45 kDa and an isoelectric pH of 4.8. The polypeptide of SEQ ID NO: 3 showed mannanase activity as shown below.

A comparative pairwise global alignment of amino acid sequences was determined using the Needleman and Wunsch algorithm (Needleman and Wunsch, 1970, J. Mol. Biol. 48: 443-453) with gap open penalty of 10, gap extension penalty of 0.5, and the EBLOSUM62 matrix. The alignment showed that the deduced amino acid sequence of the Talaromyces leycettanus gene encoding the P23YST GH5 polypeptide having mannanase activity shares 71% identity (excluding gaps) to the deduced amino acid sequence of a predicted GH5 family protein from Talaromyces stipitatus (accession number SWISSPROT:B8M6W7) with endo mannanase activity.

### Expression of the Talaromyces leycettanus GH5 Mannanase (Mannanase 1)

The expression plasmid pP23YST was transformed into Aspergillus oryzae MT3568. Aspergillus oryzae MT3568 is an AMDS (acetamidase) disrupted derivative of JaL355 (WO 02/40694) in which pyrG auxotrophy was restored in the process of knocking out the Aspergillus oryzae acetamidase (AMDS) gene. MT3568 protoplasts are prepared according to the method of European Patent No. 0238023, pages 14-15, which are incorporated herein by reference.

Transformants were purified on COVE sucrose selection plates through single conidia prior to sporulating them on PDA plates. Production of the Talaromyces leycettanus GH5 polypeptide by the transformants was analyzed from culture supernatants of 1 ml 96 deep well stationary cultivations at 30° C. in YP+2% glucose medium. Expression was verified on an E-Page 8% SDS-PAGE 48 well gel (Invitrogen, Carlsbad, CA, USA) by Coomassie staining. One transformant was selected for further work and designated Aspergillus oryzae 11.7.

For larger scale production, Aspergillus oryzae 11.7 spores were spread onto a PDA plate and incubated for five days at 37° C. The confluent spore plate was washed twice with 5 ml of 0.01% TWEEN® 20 to maximize the number of spores collected. The spore suspension was then used to inoculate fifteen 500 ml flasks containing 150 ml of Dap-4C medium (WO 2012/103350). The culture was incubated at 30° C. with constant shaking at 100 rpm. At day four post-inoculation, the culture broth was collected by filtration through a bottle top MF75 Supor MachV 0.2 μm PES filter (Thermo Fisher Scientific, Roskilde, Denmark). Fresh culture broth from this transformant produced a band of GH5 protein of approximately 42 kDa. The identity of the prominent band as the Talaromyces leycettanus GH5 polypeptide was verified by peptide sequencing.

### Alternative Method for Producing the Talaromyces leycettanus GH5 Mannanase (Mannanase 1)

Based on the nucleotide sequence identified as SEQ ID NO: 1, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, CA 94025, USA). The synthetic gene can be designed to incorporate additional DNA sequences such as restriction sites or homologous recombination regions to facilitate cloning into an expression vector.

Using the two synthetic oligonucleotide primers F-P23YST and R-P23YST described above, a simple PCR reaction can be used to amplify the full-length open reading frame from the synthetic gene of SEQ ID NO: 1. The gene can then be cloned into an expression vector for example as described above and expressed in a host cell, for example in Aspergillus oryzae as described above.

### Purification of the Talaromyces leycettanus GH5 Mannanase (Mannanase 1)

Filtrated broth was adjusted to pH7.0 and filtrated on 0.22 μm PES filter (Nalge Nunc International, Nalgene labware cat #595-4520). Following, the filtrate was added 1.8M ammonium sulphate. The filtrate was loaded onto a Phenyl Sepharose™ 6 Fast Flow column (high sub) (GE Healthcare, Piscataway, NJ, USA) equilibrated with 1.8M ammonium sulphate, 25 mM HEPES pH7.0. After wash with 1.0M ammonium sulphate, the bound proteins were batch eluted with 25 mM HEPES pH 7.0. Fractions were collected and analyzed by SDS-PAGE. The fractions were pooled and applied to a Sephadex™ G-25 (medium) (GE Healthcare, Piscataway, NJ, USA) column equilibrated in 25 mM HEPES pH 7.5. The fractions were applied to a SOURCE™ 15Q (GE Healthcare, Piscataway, NJ, USA) column equilibrated in 25 mM HEPES pH 7.5 and bound proteins were eluted with a linear gradient from 0-1000 mM sodium chloride over 20 CV. Fractions were collected and analyzed by SDS-PAGE.

### Example 2. Cloning, Expression and Purification of the Chaetomium virescens Endo-Mannanase (Mannanase 2)

### Strains

Chaetomium virescens CBS547.75 was used as the source of a polypeptide having mannanase activity. Aspergillus oryzae MT3568 strain was used for expression of the Chaetomium virescens gene encoding the polypeptide having mannanase activity. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene.

### Source of DNA Sequence Information for Chaetomium virescens Strain CBS547.75

Genomic sequence information was generated by Illumina DNA sequencing at The National Center for Genome Resources in Santa Fe, New Mexico from genomic DNA isolated from Chaetomium virescens Strain CBS547.75. A preliminary assembly of the genome was analyzed using the Abyss 1.2.0 Sequence Assembler (GSC Software Center, Vancouver, Canada). Gene models constructed by the software were used as a starting point for detecting GH5 homologues in the genome. More precise gene models were constructed manually using multiple known GH5 protein sequences as a guide.

### Chaetomium virescens Strain CBS547.75 Genomic DNA Extraction

To generate genomic DNA for PCR amplification, Chaetomium virescens Strain CBS547.75 was propagated on PDA agar plates by growing at 26° C. for 7 days. Spores harvested from the PDA plates were used to inoculate 25 ml of YP+2% glucose medium in a baffled shake flask and incubated at 26° C. for 72 hours with agitation at 85 rpm.

Genomic DNA was isolated according to a modified DNeasy Plant Maxi kit protocol (Qiagen Danmark, Copenhagen, Denmark). The fungal material from the above culture was harvested by centrifugation at 14,000×g for 2 minutes. The supernatant was removed and the 0.5 g of the pellet was frozen in liquid nitrogen with quartz sand and grinded to a fine powder in a pre-chilled mortar. The powder was transferred to a 15 ml centrifuge tube and added 5 ml buffer AP1 (preheated to 65° C.) and 10 μl RNase A stock solution (100 mg/ml) followed by vigorous vortexing. After incubation for 10 minutes at 65° C. with regular inverting of the tube, 1.8 ml buffer AP2 was added to the lysate by gentle mixing followed by incubation on ice for 10 min. The lysate was then centrifugated at 3000×g for 5 minutes at room temperature and the supernatant was decanted into a QIAshredder maxi spin column placed in a 50 ml collection tube. This was followed by centrifugation at 3000×g for 5 minutes at room temperature. The flow-through was transferred into a new 50 ml tube and added 1.5 volumes of buffer AP3/E followed by vortexing. 15 ml of the sample was transferred into a DNeasy Maxi spin column placed in a 50 ml collection tube and centrifuged at 3000×g for 5 minutes at room temperature. The flow-through was discarded and 12 ml buffer AW was added to the DNeasy Maxi spin column placed in a 50 ml collection tube and centrifuged at 3000×g for 10 minutes at room temperature. After discarding the flow-through, centrifugation was repeated to dispose of the remaining alcohol. The DNeasy Maxi spin column was transferred to a new 50 ml tube and 0.5 ml buffer AE (preheated to 70° C.) was added. After incubation for 5 minutes at room temperature, the sample was eluded by centrifugation at 3000×g for 5 minutes at room temperature. Elution was repeated with an additional 0.5 ml buffer AE and the eluates were combined. The concentration of the harvested DNA was measured by a UV spectrophotometer at 260 nm.

### Construction of an Aspergillus oryzae Expression Vector Containing Chaetomium virescens Strain CBS547.75 Genomic Sequence Encoding a Family GH5 Polypeptide Having Mannanase Activity

Two synthetic oligonucleotide primers shown below were designed to PCR amplify the Chaetomium virescens Strain CBS547.75 P23NUR gene (SEQ ID NO: 6) from the genomic DNA prepared as described above. An IN-FUSION™ Cloning Kit (BD Biosciences, Palo Alto, CA, USA) was used to clone the fragment directly into the expression vector pDau109 (WO 2005/042735).

F-P23NUR
(SEQ ID NO: 9)
5′-acacaactggggatccaccATGAAGGCAATCCTCACAGCC-3′
R-P23NUR
(SEQ ID NO: 10)
5′-ccctctagatctcgagTGCGTATCACGGGACTTCAGA-3′

Capital letters represent gene sequence. The underlined sequence is homologous to the insertion sites of pDau109.

An MJ Research PTC-200 DNA engine was used to perform the PCR reaction. A Phusion® High-Fidelity PCR Kit (Finnzymes Oy, Espoo, Finland) was used for the PCR amplification. The PCR reaction was composed of 5 μl of 5×HF buffer (Finnzymes Oy, Espoo, Finland), 0.5 μl of dNTPs (10 mM), 0.5 μl of Phusion® DNA polymerase (0.2 units/μl) (Finnzymes Oy, Espoo, Finland), 2 μl of primer F-P23NUR (2.5 M), 2 μl of primer R-P23NUR (2.5 μM), 0.5 μl of Chaetomium virescens genomic DNA (100 ng/μl), and 14.5 μl of deionized water in a total volume of 25 μl. The PCR conditions were 1 cycle at 95° C. for 2 minutes. 35 cycles each at 98° C. for 10 seconds, 60° C. for 30 seconds, and 72° C. for 2.5 minutes; and 1 cycle at 72° C. for 10 minutes. The sample was then held at 12° C. until removed from the PCR machine.

The reaction products were isolated by 1.0% agarose gel electrophoresis using 40 mM Tris base, 20 mM sodium acetate, 1 mM disodium EDTA (TAE) buffer where a 1288 bp product band was excised from the gel and purified using an illustra GFX® PCR DNA and Gel Band Purification Kit (GE Healthcare Life Sciences, Brondby, Denmark) according to the manufacturer's instructions. The fragment was then cloned into Bam HI and Xho I digested pDau109 using an IN-FUSION™ Cloning Kit resulting in plasmid pP23NUR. Cloning of the P23NUR gene into Bam HI-Xho I digested pDau109 resulted in the transcription of the Chaetomium virescens P23NUR gene under the control of a NA2-tpi double promoter. NA2-tpi is a modified promoter from the gene encoding the Aspergillus niger neutral alpha-amylase in which the untranslated leader has been replaced by an untranslated leader from the gene encoding the Aspergillus nidulans triose phosphate isomerase.

The cloning protocol was performed according to the IN-FUSION™ Cloning Kit instructions generating a P23NUR GH5 construct. The treated plasmid and insert were transformed into One Shot® TOP10F′ Chemically Competent E. coli cells (Invitrogen, Carlsbad, CA, USA) according to the manufacturer's protocol and plated onto LB plates supplemented with 0.1 mg of ampicillin per ml. After incubating at 37° C. overnight, colonies were seen growing under selection on the LB ampicillin plates. Four colonies transformed with the P23NUR GH5 construct were cultivated in LB medium supplemented with 0.1 mg of ampicillin per ml and plasmid was isolated with a QIAprep Spin Miniprep Kit (QIAGEN Inc., Valencia, CA, USA) according to the manufacturer's protocol.

Isolated plasmids were sequenced with vector primers and P23NUR gene specific primers in order to determine a representative plasmid expression clone that was free of PCR errors.

### Characterization of the Chaetomium virescens CBS547.75 Genomic Sequence Encoding a P23NUR GH5 Polypeptide Having Mannanase Activity

DNA sequencing of the Chaetomium virescens CBS547.75 P23NUR GH5 genomic clone was performed with an Applied Biosystems Model 3700 Automated DNA Sequencer using version 3.1 BIG-DYE™ terminator chemistry (Applied Biosystems, Inc., Foster City, CA, USA) and primer walking strategy. Nucleotide sequence data were scrutinized for quality and all sequences were compared to each other with assistance of PHRED/PHRAP software (University of Washington, Seattle, WA, USA).

The nucleotide sequence and deduced amino acid sequence of the Chaetomium virescens P23NUR gene is shown in SEQ ID NO: 6 and SEQ ID NO: 7, respectively. The coding sequence is 1222 bp including the stop codon and is interrupted by two introns. The encoded predicted protein is 367 amino acids. Using the SignalP program (Nielsen et al., 1997, Protein Engineering 10: 1-6), a signal peptide of 17 residues was predicted. The predicted mature protein (SEQ ID NO: 8) contains 350 amino acids with a predicted molecular mass of 39 kDa and an isoelectric pH of 6.9. The polypeptide of SEQ ID NO: 8 showed mannanase activity as shown below.

A comparative pairwise global alignment of amino acid sequences was determined using the Needleman and Wunsch algorithm (Needleman and Wunsch, 1970, J. Mol. Biol. 48: 443-453) with gap open penalty of 10, gap extension penalty of 0.5, and the EBLOSUM62 matrix. The alignment showed that the deduced amino acid sequence of the Chaetomium virescens gene encoding the P23NUR GH5 polypeptide having mannanase activity shares 86% identity (excluding gaps) to the deduced amino acid sequence of a predicted GH5 family protein from Chaetomium globosum (accession number SWISSPROT:Q2H1Y9) with unknown activity.

### Expression of the Chaetomium virescens GH5 Mannanase P23NUR

The expression plasmid pP23NUR was transformed into Aspergillus oryzae MT3568. Aspergillus oryzae MT3568 is an AMDS (acetamidase) disrupted derivative of JaL355 (WO 02/40694) in which pyrG auxotrophy was restored in the process of knocking out the Aspergillus oryzae acetamidase (AMDS) gene. MT3568 protoplasts are prepared according to the method of European Patent No. 0238023, pages 14-15, which are incorporated herein by reference.

Transformants were purified on COVE sucrose selection plates through single conidia prior to sporulating them on PDA plates. Production of the Chaetomium virescens GH5 polypeptide by the transformants was analyzed from culture supernatants of 1 ml 96 deep well stationary cultivations at 30° C. in YP+2% glucose medium. Expression was verified on an E-Page 8% SDS-PAGE 48 well gel (Invitrogen, Carlsbad, CA, USA) by Coomassie staining. One transformant was selected for further work and designated Aspergillus oryzae 29.8.

For larger scale production, Aspergillus oryzae 29.8 spores were spread onto a PDA plate and incubated for five days at 37° C. The confluent spore plate was washed twice with 5 ml of 0.01% TWEEN® 20 to maximize the number of spores collected. The spore suspension was then used to inoculate fifteen 500 ml flasks containing 150 ml of Dap-4C medium (WO 2012/103350). The culture was incubated at 30° C. with constant shaking at 100 rpm. At day four post-inoculation, the culture broth was collected by filtration through a bottle top MF75 Supor MachV 0.2 μm PES filter (Thermo Fisher Scientific, Roskilde, Denmark). Fresh culture broth from this transformant produced two bands of GH5 protein of approximately 45 and 50 kDa. The identity of the two bands as the Chaetomium virescens GH5 polypeptide was verified by peptide sequencing. The difference between apparent and observed size of the recombinant proteins can likely be attributed to glycosylation and/or other posttranslational modifications.

### Alternative Method for Producing the Chaetomium virescens GH5 Mannanase (Mannanase 2)

Based on the nucleotide sequence identified as SEQ ID NO: 6, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, CA 94025, USA). The synthetic gene can be designed to incorporate additional DNA sequences such as restriction sites or homologous recombination regions to facilitate cloning into an expression vector.

Using the two synthetic oligonucleotide primers F-P23NUR and R-P23NUR described above, a simple PCR reaction can be used to amplify the full-length open reading frame from the synthetic gene of SEQ ID NO: 4. The gene can then be cloned into an expression vector for example as described above and expressed in a host cell, for example in Aspergillus oryzae as described above.

### Purification of the Chaetomium virescens Endo-Mannanase (Mannanase 2)

Filtrated broth was adjusted to pH7.0 and filtrated on 0.22 μm PES filter (Nalge Nunc International, Nalgene labware cat #595-4520). Following, the filtrate was added 1.8M ammonium sulphate. The filtrate was loaded onto a Phenyl Sepharose™ 6 Fast Flow column (high sub) (GE Healthcare, Piscataway, NJ, USA) equilibrated with 1.8M ammonium sulphate, 25 mM HEPES pH7.0. After wash with 1.0M ammonium sulphate, the bound proteins were batch eluted with 25 mM HEPES pH 7.0. Fractions were collected and analyzed by SDS-PAGE. The fractions were pooled and applied to a Sephadex™ G-25 (medium) (GE Healthcare, Piscataway, NJ, USA) column equilibrated in 12.5 mM acetic acid pH 4.3 adjusted with NaOH. The fractions were applied to a SOURCE™ 15S (GE Healthcare, Piscataway, NJ, USA) column equilibrated in 12.5 mM acetic acid pH 4.3/NaOH and bound proteins were eluted with a linear gradient from 0-1000 mM sodium chloride over 20 CV. Fractions were collected and analyzed by SDS-PAGE.

### Example 3: Cloning, Expression and Purification of the Sordaria macrospora Endo-Mannanase (Mannanase 3)

### Strains

Sordaria macrospora DSM997 was used as the source of a polypeptide having mannanase activity. Aspergillus oryzae MT3568 strain was used for expression of the Sordaria macrospora gene encoding the polypeptide having mannanase activity. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene.

### Sordaria macrospora Strain DSM997 Genomic DNA Extraction

To generate genomic DNA for PCR amplification, Sordaria macrospora Strain DSM997 was propagated on PDA agar plates by growing at 26° C. for 7 days. Spores harvested from the PDA plates were used to inoculate 25 ml of YP+2% glucose medium in a baffled shake flask and incubated at 26° C. for 72 hours with agitation at 85 rpm.

Genomic DNA was isolated according to a modified DNeasy Plant Maxi kit protocol (Qiagen Danmark, Copenhagen, Denmark). The fungal material from the above culture was harvested by centrifugation at 14,000×g for 2 minutes. The supernatant was removed and the 0.5 g of the pellet was frozen in liquid nitrogen with quartz sand and grinded to a fine powder in a pre-chilled mortar. The powder was transferred to a 15 ml centrifuge tube and added 5 ml buffer AP1 (preheated to 65° C.) and 10 μl RNase A stock solution (100 mg/ml) followed by vigorous vortexing. After incubation for 10 minutes at 65° C. with regular inverting of the tube, 1.8 ml buffer AP2 was added to the lysate by gentle mixing followed by incubation on ice for 10 min. The lysate was then centrifugated at 3000×g for 5 minutes at room temperature and the supernatant was decanted into a QIAshredder maxi spin column placed in a 50 ml collection tube. This was followed by centrifugation at 3000×g for 5 minutes at room temperature. The flow-through was transferred into a new 50 ml tube and added 1.5 volumes of buffer AP3/E followed by vortexing. 15 ml of the sample was transferred into a DNeasy Maxi spin column placed in a 50 ml collection tube and centrifuged at 3000×g for 5 minutes at room temperature. The flow-through was discarded and 12 ml buffer AW was added to the DNeasy Maxi spin column placed in a 50 ml collection tube and centrifuged at 3000×g for 10 minutes at room temperature. After discarding the flow-through, centrifugation was repeated to dispose of the remaining alcohol. The DNeasy Maxi spin column was transferred to a new 50 ml tube and 0.5 ml buffer AE (preheated to 70° C.) was added. After incubation for 5 minutes at room temperature, the sample was eluded by centrifugation at 3000×g for 5 minutes at room temperature. Elution was repeated with an additional 0.5 ml buffer AE and the eluates were combined. The concentration of the harvested DNA was measured by a UV spectrophotometer at 260 nm.

### Construction of an Aspergillus oryzae Expression Vector Containing Sordaria macrospora Strain DSM997 Genomic Sequence Encoding a Family GH5 Polypeptide Having Mannanase Activity

Two synthetic oligonucleotide primers shown below were designed to PCR amplify the Sordaria macrospora Strain DSM997 P2453A gene (SEQ ID NO: 11) from the genomic DNA prepared as described above. P2453A correspond to the genome sequence of SwissProt entry D1ZM91, annotated as a GH5 putative cellulase. An IN-FUSION™ Cloning Kit (BD Biosciences, Palo Alto, CA, USA) was used to clone the fragment directly into the expression vector pDau109 (WO 2005/042735).

F-P2453A
(SEQ ID NO: 14)
5′-acacaactggggatccaccATGAAGTCCTTGTTCACCCTCGCC-3′
R-P2453A
(SEQ ID NO: 15)
5′-ccctctagatctcgagGTACGCAGCCACGGCGACA-3′

Capital letters represent gene sequence. The underlined sequence is homologous to the insertion sites of pDau109.

An MJ Research PTC-200 DNA engine was used to perform the PCR reaction. A Phusion® High-Fidelity PCR Kit (Finnzymes Oy, Espoo, Finland) was used for the PCR amplification. The PCR reaction was composed of 5 μl of 5×HF buffer (Finnzymes Oy, Espoo, Finland), 0.5 μl of dNTPs (10 mM), 0.5 μl of Phusion® DNA polymerase (0.2 units/μl) (Finnzymes Oy, Espoo, Finland), 2 μl of primer F-P2453A (2.5 μM), 2 μl of primer R-P2453A (2.5 M), 0.5 μl of Sordaria macrospora genomic DNA (100 ng/μl), and 14.5 μl of deionized water in a total volume of 25 μl. The PCR conditions were 1 cycle at 95° C. for 2.5 minutes. 35 cycles each at 98° C. for 10 seconds, 60° C. for 30 seconds, and 72° C. for 2.5 minutes; and 1 cycle at 72° C. for 10 minutes. The sample was then held at 12° C. until removed from the PCR machine.

The reaction products were isolated by 1.0% agarose gel electrophoresis using 40 mM Tris base, 20 mM sodium acetate, 1 mM disodium EDTA (TAE) buffer where a 1260 bp product band was excised from the gel and purified using an illustra GFX® PCR DNA and Gel Band Purification Kit (GE Healthcare Life Sciences, Brondby, Denmark) according to the manufacturer's instructions. The fragment was then cloned into Bam HI and Xho I digested pDau109 using an IN-FUSION™ Cloning Kit resulting in plasmid pP2453A. Cloning of the P2453A gene into Bam HI-Xho I digested pDau109 resulted in the transcription of the Sordaria macrospora P2453A gene under the control of a NA2-tpi double promoter. NA2-tpi is a modified promoter from the gene encoding the Aspergillus niger neutral alpha-amylase in which the untranslated leader has been replaced by an untranslated leader from the gene encoding the Aspergillus nidulans triose phosphate isomerase.

The cloning protocol was performed according to the IN-FUSION™ Cloning Kit instructions generating a P2453A GH5 construct. The treated plasmid and insert were transformed into One Shot® TOP10F′ Chemically Competent E. coli cells (Invitrogen, Carlsbad, CA, USA) according to the manufacturer's protocol and plated onto LB plates supplemented with 0.1 mg of ampicillin per ml. After incubating at 37° C. overnight, colonies were seen growing under selection on the LB ampicillin plates. Two colonies transformed with the P2453A GH5 construct were cultivated in LB medium supplemented with 0.1 mg of ampicillin per ml and plasmid was isolated with a QIAprep Spin Miniprep Kit (QIAGEN Inc., Valencia, CA, USA) according to the manufacturer's protocol.

Isolated plasmids were sequenced with vector primers and P2453A gene specific primers in order to determine a representative plasmid expression clone that was free of PCR errors.

### Characterization of the Sordaria macrospora DSM997 Genomic Sequence Encoding a P2453A GH5 Polypeptide Having Mannanase Activity

DNA sequencing of the Sordaria macrospora DSM997 P2453A GH5 genomic clone was performed with an Applied Biosystems Model 3700 Automated DNA Sequencer using version 3.1 BIG-DYE™ terminator chemistry (Applied Biosystems, Inc., Foster City, CA, USA) and primer walking strategy. Nucleotide sequence data were scrutinized for quality and all sequences were compared to each other with assistance of PHRED/PHRAP software (University of Washington, Seattle, WA, USA).

The nucleotide sequence and deduced amino acid sequence of the Sordaria macrospora P2453A gene is shown in SEQ ID NO: 11 and SEQ ID NO: 12, respectively. The coding sequence is 1203 bp including the stop codon and is interrupted by two introns. The encoded predicted protein is 361 amino acids. Using the SignalP program (Nielsen et al., 1997, Protein Engineering 10: 1-6), a signal peptide of 17 residues was predicted. The predicted mature protein contains 344 amino acids (SEQ ID NO: 13) with a predicted molecular mass of 38 kDa and an isoelectric pH of 6.4. The polypeptide of SEQ ID NO: 13 showed mannanase activity as shown below.

A comparative pairwise global alignment of amino acid sequences was determined using the Needleman and Wunsch algorithm (Needleman and Wunsch, 1970, J. Mol. Biol. 48: 443-453) with gap open penalty of 10, gap extension penalty of 0.5, and the EBLOSUM62 matrix. The alignment showed that the deduced amino acid sequence of the Sordaria macrospora gene encoding the P2453A GH5 polypeptide having mannanase activity shares 77% identity (excluding gaps) to the deduced amino acid sequence of a predicted GH5 family protein from Chaetomium globosum (accession number SWISSPROT:Q2H1Y9) with unknown activity.

### Expression of the Sordaria macrospora GH5 Mannanase (Mannanase 3)

The expression plasmid pP2453A was transformed into Aspergillus oryzae MT3568. Aspergillus oryzae MT3568 is an AMDS (acetamidase) disrupted derivative of JaL355 (WO 02/40694) in which pyrG auxotrophy was restored in the process of knocking out the Aspergillus oryzae acetamidase (AMDS) gene. MT3568 protoplasts are prepared according to the method of European Patent No. 0238023, pages 14-15, which are incorporated herein by reference.

Transformants were purified on COVE sucrose selection plates through single conidia prior to sporulating them on PDA plates. Production of the Sordaria macrospora GH5 polypeptide by the transformants was analyzed from culture supernatants of 1 ml 96 deep well stationary cultivations at 30° C. in YP+2% glucose medium. Expression was verified on an E-Page 8% SDS-PAGE 48 well gel (Invitrogen, Carlsbad, CA, USA) by Coomassie staining. One transformant was selected for further work and designated Aspergillus oryzae 46.7.

For larger scale production, Aspergillus oryzae 46.7 spores were spread onto a PDA plate and incubated for five days at 37° C. The confluent spore plate was washed twice with 5 ml of 0.01% TWEEN® 20 to maximize the number of spores collected. The spore suspension was then used to inoculate fifteen 500 ml flasks containing 150 ml of Dap-4C medium (WO 2012/103350). The culture was incubated at 30° C. with constant shaking at 100 rpm. At day four post-inoculation, the culture broth was collected by filtration through a bottle top MF75 Supor MachV 0.2 μm PES filter (Thermo Fisher Scientific, Roskilde, Denmark). Fresh culture broth from this transformant produced two bands of GH5 protein of approximately 47 and 50 kDa. The identity of the two bands as the Sordaria macrospora GH5 polypeptide was verified by peptide sequencing. The difference between apparent and observed size of the recombinant proteins can likely be attributed to glycosylation and/or other posttranslational modifications.

### Alternative Method for Producing the Sordaria macrospora GH5 Mannanase (Mannanase 3)

Based on the nucleotide sequence identified as SEQ ID NO: 11, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, CA 94025, USA). The synthetic gene can be designed to incorporate additional DNA sequences such as restriction sites or homologous recombination regions to facilitate cloning into an expression vector.

Using the two synthetic oligonucleotide primers F-P2453A and R-P2453A described above, a simple PCR reaction can be used to amplify the full-length open reading frame from the synthetic gene of SEQ ID NO: 7. The gene can then be cloned into an expression vector for example as described above and expressed in a host cell, for example in Aspergillus oryzae as described above.

### Purification of the Sordaria macrospora Endo-Mannanase (Mannanase 3)

Filtrated broth was adjusted to pH7.0 and filtrated on 0.22 μm PES filter (Nalge Nunc International, Nalgene labware cat #595-4520). Following, the filtrate was added 1.8M ammonium sulphate. The filtrate was loaded onto a Phenyl Sepharose™ 6 Fast Flow column (high sub) (GE Healthcare, Piscataway, NJ, USA) equilibrated with 1.8M ammonium sulphate, 25 mM HEPES pH7.0. After wash with 1.0M ammonium sulphate, the bound proteins were batch eluted with 12.5 mM HEPES pH 7.0. Fractions were collected and analyzed by SDS-PAGE. The fractions were pooled and applied to a Sephadex™ G-25 (medium) (GE Healthcare, Piscataway, NJ, USA) column equilibrated in 25 mM HEPES pH 7.0. The fractions were applied to a SOURCE™ 15Q (GE Healthcare, Piscataway, NJ, USA) column equilibrated in 12.5 mM HEPES PH 7.5 and bound proteins were eluted with a linear gradient from 0-1000 mM sodium chloride over 20CV. Fractions were collected and analyzed by SDS-PAGE. The protein was recovered in the effluent.

### Example 4: Thermostability of Mannanases Evaluated by DSC

MANNANASE 4 used in this and some of the following examples is a GH5_8 mannanase originally obtained from Caldicellulosiruptor saccharolyticus and having an amino acid sequence represented by the mature amino acid sequence of SEQ ID NO: 17. The mature amino acid sequence has been determined as amino acids 28-319 by N-terminal sequencing and mass spectrometry (MS) of the full-length protein. The mature amino acid sequence is shown as SEQ ID NO: 18.

Thermostabilities of MANNANASE 1, MANNANASE 2, MANNANASE 3 and MANNANASE 4 were evaluated by Differential Scanning Calorimetry (DSC) in the appropriate buffer solution (20 mM Sodium acetate pH 5). The temperature corresponding to the apex of the peak in the thermogram was noted as the thermal transition midpoint (Tₘ (° C.)) for the enzymes.

TABLE 1
Midpoint temperatures

| Enzyme | Temperature (° C.) |
|---|---|
| MANNANASE 1 | 93 |
| MANNANASE 2 | 69 |
| MANNANASE 3 | 74 |
| MANNANASE 4 | 92 |

For comparison, the thermal transition midpoint for Mannaway determined by DSC at pH 5 is 73° C. The thermal transition midpoint for Gamanase (beta-mannanase from Aspergillus niger) determined by DSC at pH 5 is 87° C. And the thermal transition midpoint for beta-mannanase from Trichoderma reesei used in Example 10 determined by DSC at pH 5 is 81° C.

### Example 5: Mannanase Activity on AZCL-Galactomannan

Activity of the mannanases were assayed by the hydrolysis of 0.2 w/v % AZCL-galactomannan in 50 mM Britton-Robinson Buffer (50 mM phosphoric acid, 50 mM acetic acid, 50 mM boric acid, 50 mM KCl, 1 mM CaCl₂) and 0.01% Triton X-100, pH 5 at 40° C. for 10 min. Experimental mannanases and Mannaway® 25L were added individually to give a final concentration of 0-0.01 mg/ml. The reactions were terminated on an ice/water bath. After centrifugation (10,000 rpm, 5 min at 4° C.), the supernatants were transferred to a microtiter plate and the absorbance at 595 nm was measured. The procedures were performed in triplicates for all enzymes and a blank (no enzyme). For all 4 enzymes a dose response could be observed (Table 2).

TABLE 2
Absorbance at 600 nm.

| | | MANNA- | MANNA- | MANNA- |
|---|---|---|---|---|
| Conc. | Mannaway | NASE 1 | NASE 2 | NASE3 |
| (mg/mL) | A₆₀₀ | A₆₀₀ | A₆₀₀ | A₆₀₀ |
| 0.0100 | 0.529 | 1.201 | 0.463 | 0.294 |
| 0.0075 | 0.405 | 1.034 | 0.381 | 0.233 |
| 0.0050 | 0.282 | 0.811 | 0.295 | 0.163 |
| 0.0025 | 0.156 | 0.535 | 0.178 | 0.087 |
| 0.0010 | 0.070 | 0.257 | 0.082 | 0.037 |
| 0.0000 | 0.000 | 0.000 | 0.000 | 0.000 |

### Example 6: Pretreatment of Coffee Material

800 mL boiling water was added to 155 g roasted and grinded Arabica coffee beans with a particle size of 0.5 mm. After incubation in a water bath at 95° C. for 30 min with manual mixing every 5 min, the slurry was cooled down at room temperature. After an initial vacuum filtration through a Whatman GF/D filter, ∅150 mm, the insoluble spent coffee on the filter was washed by adding 500-1000 mL MilliQ water. The spent coffee was removed from the filter and spread out on a large sheet and left to dry overnight. The spent coffee grounds were further defatted by water saturated butanol. The butanol fraction was separated by filtration and the defatted spent coffee grounds were dried under vacuum before use. This defatted spent coffee grounds were used in Example 7.

### Example 7: Enzyme Catalyzed Hydrolysis of Defatted Spent Coffee Grounds

### a) Enzymatic Extraction

Defatted spent coffee grounds produced according to Example 6 (10 weight %) was incubated with water and a suitably diluted enzyme (to give a final reaction concentration of 1.47 nM for MANNANASE 1-4 and 0.2% Mannaway® 25 L) at 50° C. Samples were withdrawn after 2 and 24 hours and the enzymatic hydrolysis was stopped immediately by heating the samples at 100° C. for 10 min. After centrifugation (10,000×g, 10 min) and filtration through a 0.22 μm filter, the supernatants were further analyzed for dry matter, carbohydrate composition and absorbance. The procedures were performed in duplicate for all enzymes and a blank (no enzyme added).

### b) Dry Matter Determination

Dry matter (DM) content was quantified after overnight drying at 110° C. of supernatants from enzyme treated spent coffee grounds. The weight of the dry matter was divided by the added volume of supernatant and a DM value based on g/L was calculated. The characteristics of the extract based on DM are summarized in Table 3.

TABLE 3
Dry matter of defatted spent grounds extract
after different enzymatic treatments.

| | Dry matter (g/L) | |
|---|---|---|
| Enzyme treatment | 2 h | 24 h |
| No enzyme | 2.8 | 2.5 |
| Mannaway | 6.1 | 7.2 |
| MANNANASE 1 | 10.0 | 16.1 |
| MANNANASE 2 | 9.6 | 11.3 |
| MANNANASE 3 | 8.0 | 9.1 |
| MANNANASE 4 | 8.1 | 9.2 |

All experimental mannanases solubilize more dry matter than Mannaway, both after 2 hrs and 24 hrs incubation time (Table 3).

### c) Carbohydrate Analysis

The sugar composition was analysed by measuring free monosaccharides in the supernatants by high-performance anion exchange chromatography with pulsed amperometric detection (HPAEC-PAD). The total sugars were analysed by HPAEC-PAD after acid hydrolysis in 2 M trifluoro acetic acid for 2 h at 95° C. The acid hydrolysed samples were neutralised by an initial dilution in 0.2 M NaOH. Monosaccharides were quantified after suitable dilutions against a 5-point standard curve of arabinose (Ara), galactose (Gal), glucose (Glc) and mannose (Man) between 0.002-0.02 g/L. The results can be seen in Table 4, Table 5 and Table 6.

TABLE 4
Free monosaccharides in the extract from defatted
spent coffee grounds after enzymatic treatment.

| | Free monosaccharides/DM (%) | | | |
|---|---|---|---|---|
| | 2 h | | 24 h | |
| Enzyme treatment | Glu | Man | Glu | Man |
| No enzyme | 0 | 0 | 0 | 0 |
| Mannaway | 0 | 1.5 | 0 | 1.5 |
| MANNANASE 1 | 0.2 | 2.0 | 0.2 | 4.6 |
| MANNANASE 2 | 0 | 1.6 | 0 | 2.3 |
| MANNANASE 3 | 0 | 0.4 | 0 | 0.5 |

TABLE 5
Total sugar composition in the extract from defatted spent coffee
grounds after enzymatic treatment. Monosaccharides in
supernatant after acid hydrolysis.

| | Total monosaccharides/DM (%) | | | | | | | |
|---|---|---|---|---|---|---|---|---|
| | 2 h | | | | 24 h | | | |
| Enzyme treatment | Ara | Gal | Glu | Man | Ara | Gal | Glu | Man |
| No enzyme | 0.7 | 2.4 | 0.8 | 11.0 | 1.7 | 6.1 | 0.9 | 15.2 |
| Mannaway | 0.7 | 2.7 | 1.1 | 19.6 | 0.9 | 3.9 | 0.6 | 21.3 |
| MANNANASE 1 | 0.5 | 2.4 | 1.3 | 18.7 | 0.6 | 3.2 | 0.4 | 20.0 |
| MANNANASE 2 | 0.5 | 2.5 | 1.1 | 18.9 | 0.7 | 3.5 | 0.4 | 20.4 |
| MANNANASE 3 | 0.5 | 2.6 | 1.1 | 18.3 | 0.8 | 3.6 | 0.4 | 19.8 |

TABLE 6
Percentage of the saccharides present as monosaccharides
based on the weight of the total sugars as monosaccharides.

| | Free monosaccharides/ | |
|---|---|---|
| | Total sugars (%) | |
| Enzyme treatment | 2 h | 24 h |
| No enzyme | 0 | 0 |
| Mannaway | 6 | 6 |
| MANNANASE 1 | 10 | 20 |
| MANNANASE 2 | 7 | 9 |
| MANNANASE 3 | 2 | 2 |

### d) Absorbance

The absorbance at 361 nm of samples was measured after suitable dilutions of supernatants and alkalinisation by at least a 1:10 dilution in 0.2 M Na₂CO₃. Dividing the absorbance by the DM (g/L) gave a quality measurement relating to released colour by DM (Table 7). The mannanases released similar colour per DM as Mannaway.

TABLE 7
Quality of extract. Absorbance of extract after
alkalinisation at 361 nm per dry matter.

| | Absorbance | |
|---|---|---|
| | (A₃₆₁ * L/g) | |
| Enzyme treatment | 2 h | 24 h |
| Mannaway | 0.04 | 0.05 |
| MANNANASE 1 | 0.04 | 0.04 |
| MANNANASE 2 | 0.04 | 0.03 |
| MANNANASE 3 | 0.04 | 0.04 |

### Example 8: Enzyme Catalyzed Hydrolysis of Defatted Spent Coffee Grounds

The enzymatic solubilisation of spent defatted coffee grounds were performed with Mannaway and MANNANASE4 using the method describe in Example 7. The difference was that two temperatures were tested for the enzymatic extraction, 50° C. and 80° C., and dry matter was measured on the resulting supernatants.

TABLE 8
Dry matter of defatted spent grounds extract after
different enzymatic treatments at 50° C. and 80° C.

| | Dry matter (g/L) | | | |
|---|---|---|---|---|
| | 50° C. | | 80° C. | |
| Enzyme treatment | 2 h | 24 h | 2 h | 24 h |
| No enzyme | 1.2 | 0.5 | 1.4 | 3.2 |
| Mannaway | 5.8 | 6.7 | 4.6 | 3.6 |
| MANNANASE 4 | 8.1 | 9.2 | 8.8 | 11.4 |

Table 8 clearly shows that using a thermostable mannanase enables higher solubilisation temperatures and achieves higher solubilisation degrees at equal enzyme dosing.

### Example 9: Enzyme Catalyzed Hydrolysis of Spent Coffee Grounds

### Preparation of Spent Coffee

Roasted Arabica beans (238 g) were milled using a 1 mm sieve and the resulting milled fraction was extracted with boiling water at a dry matter of 20%. The temperature after mixing was 87° C. The spent coffee grounds were separated from the liquid by vacuum filtration using Whatman GF/D filters after 10 min of mixing. The spent coffee was washed with an excess of water before drying over night at 60° C. Based on the dry matter in the filtrate the partition of the dry matter was 25% in the liquid phase and 75% in the solid phase.

### Enzymatic Hydrolysis of the Spent Coffee Produced

Spent coffee grounds produced as described above was incubated at 10 weight % with water and a suitably diluted mannanase (to give a final reaction concentration of 50 mg/L for MANNANASE 1, MANNANASE 4 and 0.2 volume % Mannaway® 25 L) at 50, 70, 80 and 90° C. Samples were heat inactivated at 100° C. for 10 min after 2 or 24 hours enzymatic hydrolysis. After centrifugation (10,000×g, 10 min) and filtration through a 0.22 μm filter, the supernatants were analyzed for dry matter. The procedures were performed in duplicate for all enzymes and a blank (no enzyme added).

Dry matter was measured as described in Example 7.

TABLE 9
The effect of temperature and time on the solubilization-degree of spent coffee

| | 2 hrs | | | | 24 hrs | | | |
|---|---|---|---|---|---|---|---|---|
| Treatment | 50° C. | 70° C. | 80° C. | 90° C. | 50° C. | 70° C. | 80° C. | 90° C. |
| No enzyme | 1%¹ | 2% | 2% | 3% | 3% | 4% | 5% | 7% |
| Mannaway ® 25 L | 4% | 3% | 2% | 3% | 7% | 4% | 5% | 8% |
| MANNANASE 1 | 4% | 5% | 5% | 3% | 9% | 12% | 10% | 8% |
| MANNANASE 4 | 6% | 9% | 10% | 1%¹ | 14% | 21% | 17% | 10% |

¹Standard deviation above 1 percentage point

Mannaway could solubilize some of the spent coffee grounds at 50° C. in both the short and long incubation time but at temperatures at or above 70° C. there was no significant solubilization compared to the untreated sample. For MANNANASE 1 and MANNANASE 4 the solubilization optimum for the longer enzyme incubation was 70° C. and at the shorter incubation time 70-80° C. was the optimal temperature range (Table 9). MANNANASE 1 and MANNANASE 4 could therefore be used at higher temperature where significantly increased extraction yields were observed and hence lead to better process economy.

### Example 10

Coffee grounds prepared according to Example 6 was used at a final assay concentration of 10% dry matter and hydrolysed for 2 or 24 hrs at 55° C. on a thermomixer at 1200 rpm. The enzyme concentration was 0.5 mg enzyme per kg spent coffee grounds. The enzyme was inactivated by boiling for 10 min and the supernatant transferred to a separate tube after 10 min centrifugation at 10,000 rfc. Approximately 0.75 g extract was taken out and the liquid evaporated at 105° C. before the dry matter was recorded.

Gamanase is beta-mannanase from Aspergillus niger. “T. reesei+CBM1” is beta-mannanase from Trichoderma reesei including a CBM1 binding domain having the amino acid sequence shown as amino acids 1 to 418 of SEQ ID NO: 19. “T. reesei-CBM1” is beta-mannanase from Trichoderma reesei without the CBM1 binding domain having the amino acid sequence shown as amino acids 1 to 355 of SEQ ID NO: 20.

TABLE 10
Enzymatic solubilization of defatted coffee grounds
compared to a blank with no enzyme added

| | 2 hrs | 24 hrs |
|---|---|---|
| No enzyme added | 1.1 ± 0.2% | 3.0 ± 0.1% |
| Mannaway | 2.2 ± 0.1% | 2.7 ± 0.2% |
| MANNANASE 1 | 7.5 ± 0.1% | 14.7 ± 0.2% |
| Gamanase | 1.9 ± 0.1% | 5.2 ± 0.1% |
| T. reesei + CBM1 | 4.5 ± 0.3% | 10.3 ± 0.2% |
| T. reesei − CBM1 | 2.9 ± 0% | 8.1 ± 0.2% |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-15. (canceled)

16. A method for producing a coffee extract, comprising:
(a) incubating roasted and ground coffee beans in water with a GH5 endo-beta-1,4-mannanase, at a temperature of 50-80° C. to make an aqueous coffee extract, wherein the GH5 endo-beta-1,4-mannanase comprises an amino acid sequence with at least 90% sequence identity to SEQ ID NO: 3, has mannanase activity, and is thermostable; and
(b) separating the coffee extract from the coffee beans.

17. The method of claim 16, wherein the GH5 endo-beta-1,4-mannanase comprises an amino acid sequence with at least 95% sequence identity to SEQ ID NO: 3.

18. The method of claim 16, wherein the GH5 endo-beta-1,4-mannanase comprises an amino acid sequence with at least 97% sequence identity to SEQ ID NO: 3.

19. The method of claim 16, wherein the GH5 endo-beta-1,4-mannanase comprises the amino acid sequence of SEQ ID NO: 3.

20. The method of claim 16, wherein the GH5 endo-beta-1,4-mannanase has a melting temperature (Tₘ) determined by Differential Scanning Calorimetry (DSC) of at least 80° C.

21. The method of claim 16, wherein the incubation in step (a) is performed at a temperature of 60-80° C.

22. The method of claim 16, wherein the incubation in step (a) is performed at a temperature of 65-80° C.

23. The method of claim 16, wherein the incubation in step (a) is performed at a temperature of 70-80° C.

24. The method of claim 16, wherein the incubation in step (a) is performed at a temperature of 75-80° C.

25. The method of claim 16, wherein the incubation in step (a) is performed at a temperature of 80° C.

26. The method of claim 16, wherein the incubation in step (a) is performed for at least one hour.

27. The method of claim 16, wherein the incubation in step (a) is performed for at least two hours.

28. The method of claim 16, wherein the incubation in step (a) is performed for at least four hours.

29. The method of claim 16, wherein the incubation in step (a) is performed for 1-48 hours.

30. The method of claim 16, wherein the coffee extract obtained in step (b) comprises at least 100% more dry matter than a coffee extract prepared by a similar method without the addition of the GH5 endo-beta-1,4-mannanase which comprises an amino acid sequence with at least 90% sequence identity to SEQ ID NO: 3, has mannanase activity, and is thermostable.

31. The method of claim 16, wherein the roasted and ground coffee beans are incubated in water, the GH5 endo-beta-1,4-mannanase which comprises an amino acid sequence with at least 90% sequence identity to SEQ ID NO: 3, has mannanase activity, and is thermostable, and other enzymes selected from the group consisting of cellulases and galactanases.

32. The method of claim 16, further comprising subjecting the roast and ground coffee beans to one or more first extractions before step (a).

33. The method of claim 32, further comprising a steam explosion after the one or more first extractions and before step (a).

34. The method of claim 32, further comprising milling of the coffee beans after the one or more first extractions and before step (a).

35. The method of claim 32, wherein at least 8% by weight of the dry matter of the coffee beans obtained after the one or more first extractions is recovered in the coffee extract obtained in step (b).


SEQUENCE LISTING (20 sequences)

SEQ ID NO: 1 - DNA, 1548 bp, Talaromyces leycettanus
  intron 170..263
  intron 972..1053
  intron 1183..1258
  source 1..1548: mol_type = genomic DNA
  CDS 1..169
  CDS 264..971
  CDS 1054..1182
  CDS 1259..1545
atgaagttgtctaccctcaatttcctgtccttggccggtctggtgtctgcccaggttgccaactatggccaatgtggtggacagaattattctggcccgacaacttgcaatccgggctggtcttgccaatatctgaatccatattatagccagtgtcttccagctacccgtatgtcgactacactcatgtgcatatcagactctgatgttcccatccgcttttggtactacattcttgtttccttgctaattcatcaacacagaaacgaccactctgacgacgtcgacgaagcccaccagcaccagcaccaccaccagaaccagtaccagtaccaccagcacccagggcggctcgtcaagcacatctatacccagcaagaatggtctcaagtttaccattgacggcaagaccgcctactatgcaggcaccaacacctactggctcccgttcctgaccaacaatgcggatgttgatctggtcatgagccatctccaacaatccggcctcaagatccttcgtgtctggggcttcaacgacgtcaacacccagccaggaagtggcaccgtgtggttccagctgctccagaacggccaggcgactatcaacacgggcgccaatggtctacagcgcctcgactacgtggtgcaatctgcggaagctcacgatatcaaactgatcattaactttgtcaacaactggaacgattatggcggcatcaacgcgtacgtcaataactatggcggtaatgcaacgacctggtacaccaactcggccgctcaggctgcgtatcgtaactacatcaaggcggtcatctctcggtacattggctctcctgcgatctttgcttgggagttggccaatgagccccgctgccatgggtgcgacacctctgtgatctacaactgggtctctagcaccagtgcatacatcaagtctcttgagccaaaccgcatggtctgcatcggagatggtaagtcccccctccggggagctcgagatgacaaactcgaaacccatgattcaatcaaaactaacattcgtaatctgttcagagggcatgggtctcaccaccggatccgacggcagttatcccttccaatacaccgaaggtaccgacttcgagaagaacctggccatccccaccattgatttcggcaccctgcacttgtaccctagcagctgtaagtcaaagcctcttttccagtccatatgcatacacagaaccccttccactgactcgtacttttctccgaatagggggcgaacaagactcctggggcagcacctggatctccgcccacggccaagcatgcgtcaatgccggcaagccctgcctcctggaagaatatggatccaccaatcactgctcttccgaagctccctggcagtcgaccgctctcagcacgaacggtatcgcggctgacagtttctggcagtacggtgataccttaagcacgggccagtcgccgaatgacgggtataccatttactacggtagcagcgattatacctgcttggtgacgaatcatattagccagtttcagtga

SEQ ID NO: 2 - protein, 431 aa, Talaromyces leycettanus
MKLSTLNFLSLAGLVSAQVANYGQCGGQNYSGPTTCNPGWSCQYLNPYYSQCLPATQTTTLTTSTKPTSTSTTTRTSTSTTSTQGGSSSTSIPSKNGLKFTIDGKTAYYAGTNTYWLPFLTNNADVDLVMSHLQQSGLKILRVWGFNDVNTQPGSGTVWFQLLQNGQATINTGANGLQRLDYVVQSAEAHDIKLIINFVNNWNDYGGINAYVNNYGGNATTWYTNSAAQAAYRNYIKAVISRYIGSPAIFAWELANEPRCHGCDTSVIYNWVSSTSAYIKSLEPNRMVCIGDEGMGLTTGSDGSYPFQYTEGTDFEKNLAIPTIDFGTLHLYPSSWGEQDSWGSTWISAHGQACVNAGKPCLLEEYGSTNHCSSEAPWQSTALSTNGIAADSFWQYGDTLSTGQSPNDGYTIYYGSSDYTCLVTNHISQFQ

SEQ ID NO: 3 - protein, 414 aa, Talaromyces leycettanus
QVANYGQCGGQNYSGPTTCNPGWSCQYLNPYYSQCLPATQTTTLTTSTKPTSTSTTTRTSTSTTSTQGGSSSTSIPSKNGLKFTIDGKTAYYAGTNTYWLPFLTNNADVDLVMSHLQQSGLKILRVWGFNDVNTQPGSGTVWFQLLQNGQATINTGANGLQRLDYVVQSAEAHDIKLIINFVNNWNDYGGINAYVNNYGGNATTWYTNSAAQAAYRNYIKAVISRYIGSPAIFAWELANEPRCHGCDTSVIYNWVSSTSAYIKSLEPNRMVCIGDEGMGLTTGSDGSYPFQYTEGTDFEKNLAIPTIDFGTLHLYPSSWGEQDSWGSTWISAHGQACVNAGKPCLLEEYGSTNHCSSEAPWQSTALSTNGIAADSFWQYGDTLSTGQSPNDGYTIYYGSSDYTCLVTNHISQFQ

SEQ ID NO: 4 - DNA, 45 bp, synthetic construct
  misc_feature 1..45: note = Primer
  source 1..45: mol_type = other DNA
acacaactggggatccaccatgaagttgtctaccctcaatttcct

SEQ ID NO: 5 - DNA, 38 bp, synthetic construct
  misc_feature 1..38: note = Primer
  source 1..38: mol_type = other DNA
ccctctagatctcgagcacgtcagtatcagcgaagcat

SEQ ID NO: 6 - DNA, 1222 bp, Chaetomium virescens
  intron 800..859
  intron 950..1007
  source 1..1222: mol_type = genomic DNA
  CDS 1..799
  CDS 860..949
  CDS 1008..1219
atgaaggcaatcctcacagccgggctcggactgctctccgcagtccaggctcttccctcggcgaaggctgcctctgccaccaccaacggcactcgcttcaccgtcgacggcaagacgggctacttcgcgggtaccaattcgtactggatcggcttcctgaccaacaacaaggacatcgacaccactctggaccacatctcgtcctctggtctcaagatcctgcgcgtctggggcttcaacgacgtcaacaccaagcccagcgacggcactgtctggtaccagctcctctccccgtccggttcaaagatcaacacgggtgccgacggcctgcagcggctcgaccatgtagtcaagtccgctgagaagcgcggcgtcaagctgatcatcaacttcgtcaacaactgggacgattacggcggcatgaacgcctacgtcaaggccttcggcggcaccaaggagggttggtacaccaacgccaaggctcagcagcagtacaagaagtacatcaaggccgtggtcagccgctatgccaagtcgccagccgtgtttgcctgggagctggcgaacgagccccgctgcaagggatgcagcaccgatgtcatctacaagtgggcgaccgagatctcggcgtacatccgcaagctggatccgagccacatgatcacgctcggtgatgagggctttggcctgcctggtgacacgacctacccgtacagctacaccgagggtgtcgattttgtcaagaacttgggcatcaagaacttggactttggaacattccatatgtatcccgacagctgtgcgttgactccccgtcccttcccccatctattacctttgagactgacaggggagaaagggggcgtcccatacagcttcggcgaggggtggatcaagaaccatgccgcggcttgcaagccagccggcaagccttgtcttttggaggagtgtacgttccactaccagccctttcccagcccatgagcgaaatctgacagcccgtgcagatggtgccgaacacagctgcgacatccagaagccctggcagcaggcctcgctcgcgctcgccaaggagggcatgtcgggagacctcttctggcaatggggtgacgccctgagcttcggccagtcacccaacgacggccacacggtctactacggctcggagcttgctcaatgcctggttacagatcatgttaaggagattaatgcttcttcgtaa

SEQ ID NO: 7 - protein, 367 aa, Chaetomium virescens
MKAILTAGLGLLSAVQALPSAKAASATTNGTRFTVDGKTGYFAGTNSYWIGFLTNNKDIDTTLDHISSSGLKILRVWGFNDVNTKPSDGTVWYQLLSPSGSKINTGADGLQRLDHVVKSAEKRGVKLIINFVNNWDDYGGMNAYVKAFGGTKEGWYTNAKAQQQYKKYIKAVVSRYAKSPAVFAWELANEPRCKGCSTDVIYKWATEISAYIRKLDPSHMITLGDEGFGLPGDTTYPYSYTEGVDFVKNLGIKNLDFGTFHMYPDSWGVPYSFGEGWIKNHAAACKPAGKPCLLEEYGAEHSCDIQKPWQQASLALAKEGMSGDLFWQWGDALSFGQSPNDGHTVYYGSELAQCLVTDHVKEINASS

SEQ ID NO: 8 - protein, 350 aa, Chaetomium virescens
LPSAKAASATTNGTRFTVDGKTGYFAGTNSYWIGFLTNNKDIDTTLDHISSSGLKILRVWGFNDVNTKPSDGTVWYQLLSPSGSKINTGADGLQRLDHVVKSAEKRGVKLIINFVNNWDDYGGMNAYVKAFGGTKEGWYTNAKAQQQYKKYIKAVVSRYAKSPAVFAWELANEPRCKGCSTDVIYKWATEISAYIRKLDPSHMITLGDEGFGLPGDTTYPYSYTEGVDFVKNLGIKNLDFGTFHMYPDSWGVPYSFGEGWIKNHAAACKPAGKPCLLEEYGAEHSCDIQKPWQQASLALAKEGMSGDLFWQWGDALSFGQSPNDGHTVYYGSELAQCLVTDHVKEINASS

SEQ ID NO: 9 - DNA, 40 bp, synthetic construct
  misc_feature 1..40: note = Primer
  source 1..40: mol_type = other DNA
acacaactggggatccaccatgaaggcaatcctcacagcc

SEQ ID NO: 10 - DNA, 37 bp, synthetic construct
  misc_feature 1..37: note = Primer
  source 1..37: mol_type = other DNA
ccctctagatctcgagtgcgtatcacgggacttcaga

SEQ ID NO: 11 - DNA, 1203 bp, Sordaria macrospora
  intron 782..838
  intron 929..988
  source 1..1203: mol_type = genomic DNA
  CDS 1..781
  CDS 839..928
  CDS 989..1200
atgaagtccttgttcaccctcgccctcggcttgctatcattggtctcagctgcgcctcccactgtcaatggcacacgcttctccatcgacggcaaaacggggtactttgccggtaccaactcgtactggatcggcttcctaaccaaaaaccgagatgtcgacaccgtccttgaccacatctcctcctccggactcaaaatcctgcgcatctggggcttcaacgacgtcacccgcaagccagcctccggcaccgtgtggtaccagctcctctcctcgtccggttcccagatcaacaccggtgccgatggcctgcagcgcctggactacgtcgtccagtccgccgaaaagcgtggtgtcaagctcatcattaactttgtcaacaactggagcgactacggcggcatgccagcctacgtgactgcattcggaggttcccaggagagctggtacaccaacagtcgggcgcaggcgcagtacaaggcctacattgccgctgttgtcaaccgctatatcaactcttccgctgtctttgcctgggagctggcgaacgagccccgctgcaagggatgtagcactgatgttatttacaagtgggcaactgacatctcagcttacatccgcagtttggattgcaaccacatgatcaccctcggagacgaagggtttggacttcccggggcgaccagctatccgtatcaaaccagcgaaggcgtggattttgtcaagaatctggccattaagaacttggattttggcactttccacttctatccgcaaagctgtacgtaacatgaaactcccatgtcagggaatcaacactgaccgtgactattcttagggggggtgggcaatgctgttggtgcagcttggatcaaagaccatgcctcggcttgcaagaaggccgggaagccttgtctatttgaggagtgtaagtggatgctgtgagccagtctgatttgcaggtagctaacgaatgctattcttccagatggcacctcaaccgatcactgcaccatcgagcgaccttggcaacaagcctccctccaagctgccacggagggcatggcagctgacttgttttggcaatggggagataatctgagcacggggcagacacacaatgacgggaacacgatctattatggatcagccgatgccacttgcttgattaccgagcatgtcagggccatcaactccctctag

SEQ ID NO: 12 - protein, 361 aa, Sordaria macrospora
MKSLFTLALGLLSLVSAAPPTVNGTRFSIDGKTGYFAGTNSYWIGFLTKNRDVDTVLDHISSSGLKILRIWGFNDVTRKPASGTVWYQLLSSSGSQINTGADGLQRLDYVVQSAEKRGVKLIINFVNNWSDYGGMPAYVTAFGGSQESWYTNSRAQAQYKAYIAAVVNRYINSSAVFAWELANEPRCKGCSTDVIYKWATDISAYIRSLDCNHMITLGDEGFGLPGATSYPYQTSEGVDFVKNLAIKNLDFGTFHFYPQSWGVGNAVGAAWIKDHASACKKAGKPCLFEEYGTSTDHCTIERPWQQASLQAATEGMAADLFWQWGDNLSTGQTHNDGNTIYYGSADATCLITEHVRAINSL

SEQ ID NO: 13 - protein, 344 aa, Sordaria macrospora
APPTVNGTRFSIDGKTGYFAGTNSYWIGFLTKNRDVDTVLDHISSSGLKILRIWGFNDVTRKPASGTVWYQLLSSSGSQINTGADGLQRLDYVVQSAEKRGVKLIINFVNNWSDYGGMPAYVTAFGGSQESWYTNSRAQAQYKAYIAAVVNRYINSSAVFAWELANEPRCKGCSTDVIYKWATDISAYIRSLDCNHMITLGDEGFGLPGATSYPYQTSEGVDFVKNLAIKNLDFGTFHFYPQSWGVGNAVGAAWIKDHASACKKAGKPCLFEEYGTSTDHCTIERPWQQASLQAATEGMAADLFWQWGDNLSTGQTHNDGNTIYYGSADATCLITEHVRAINSL

SEQ ID NO: 14 - DNA, 43 bp, synthetic construct
  misc_feature 1..43: note = Primer
  source 1..43: mol_type = other DNA
acacaactggggatccaccatgaagtccttgttcaccctcgcc

SEQ ID NO: 15 - DNA, 35 bp, synthetic construct
  misc_feature 1..35: note = Primer
  source 1..35: mol_type = other DNA
ccctctagatctcgaggtacgcagccacggcgaca

SEQ ID NO: 16 - protein, 309 aa, Bacillus sp.
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSP

SEQ ID NO: 17 - protein, 335 aa, Caldicellulosiruptor saccharolyticus
MKKPLGKIVASTALLISVAFSSSIASAATSNDGVVKIDTSTLIGTNHAHCWYRDRLDTALRGIRSWGMNSVRVVLSNGYRWTKIPASEVANIISLSRSLGFKAIILEVHDTTGYGEDGAACSLAQAVEYWKEIKSVLDGNEDFVIINIGNEPYGNNNYQNWVNDTKNAIKALRDAGFKHTIMVDAPNWGQDWSNTMRDNAQSIMEADPLRNLVFSIHMYGVYNTASKVEEYIKSFVDKGLPLVIGEFGHQHTDGDPDEEAIVRYAKQYKIGLFSWSWCGNSSYVGYLDMVNNWDPNNPTPWGQWYKTNAIGTSSTPTPTSTVTPTPPPRQHQHRQ

SEQ ID NO: 18 - protein, 292 aa, Caldicellulosiruptor saccharolyticus
ATSNDGVVKIDTSTLIGTNHAHCWYRDRLDTALRGIRSWGMNSVRVVLSNGYRWTKIPASEVANIISLSRSLGFKAIILEVHDTTGYGEDGAACSLAQAVEYWKEIKSVLDGNEDFVIINIGNEPYGNNNYQNWVNDTKNAIKALRDAGFKHTIMVDAPNWGQDWSNTMRDNAQSIMEADPLRNLVFSIHMYGVYNTASKVEEYIKSFVDKGLPLVIGEFGHQHTDGDPDEEAIVRYAKQYKIGLFSWSWCGNSSYVGYLDMVNNWDPNNPTPWGQWYKTNAIGTSSTPTPT

SEQ ID NO: 19 - protein, 418 aa, Trichoderma reesei
AVLQPVPRASSFVTISGTQFNIDGKVGYFAGTNCYWCSFLTNHADVDSTFSHISSSGLKVVRVWGFNDVNTQPSPGQIWFQKLSATGSTINTGADGLQTLDYVVQSAEQHNLKLIIPFVNNWSDYGGINAYVNAFGGNATTWYTNTAAQTQYRKYVQAVVSRYANSTAIFAWELGNEPRCNGCSTDVIVQWATSVSQYVKSLDSNHLVTLGDEGLGLSTGDGAYPYTYGEGTDFAKNVQIKSLDFGTFHLYPDSWGTNYTWGNGWIQTHAAACLAAGKPCVFEEYGAQQNPCTNEAPWQTTSLTTRGMGGDMFWQWGDTFANGAQSNSDPYTVWYNSSNWQCLVKNHVDAINGGTTTPPPVSSTTTTSSRTSSTPPPPGGSCSPLYGQCGGSGYTGPTCCAQGTCIYSNYWYSQCLNT

SEQ ID NO: 20 - protein, 355 aa, Trichoderma reesei
AVLQPVPRASSFVTISGTQFNIDGKVGYFAGTNCYWCSFLTNHADVDSTFSHISSSGLKVVRVWGFNDVNTQPSPGQIWFQKLSATGSTINTGADGLQTLDYVVQSAEQHNLKLIIPFVNNWSDYGGINAYVNAFGGNATTWYTNTAAQTQYRKYVQAVVSRYANSTAIFAWELGNEPRCNGCSTDVIVQWATSVSQYVKSLDSNHLVTLGDEGLGLSTGDGAYPYTYGEGTDFAKNVQIKSLDFGTFHLYPDSWGTNYTWGNGWIQTHAAACLAAGKPCVFEEYGAQQNPCTNEAPWQTTSLTTRGMGGDMFWQWGDTFANGAQSNSDPYTVWYNSSNWQCLVKNHVDAINGGT